(19) United States
(12) Patent Application Publication
(10) Pub. No.: US 2012/0178906 A1
(43) Pub. Date: Jul. 12, 2012

(54) CHELATION OF METALS TO THIOL GROUPS USING IN SITU REDUCTION OF DISULFIDE-CONTAINING COMPOUNDS BY PHOSPHINES

(75) Inventors: Aldo Cagnolini, Edison, NJ (US); Karen E. Linder, Kingston, NJ (US); Kondareddiar Ramalingam, Stockbridge, GA (US); Hong (Helen) Fan, Shanghai PuDong (CN)

(73) Assignee: BRACCO IMAGING S.P.A., Milan (IT)

(21) Appl. No.: 13/423,751

(22) Filed: Mar. 19, 2012

Related U.S. Application Data
(62) Division of application No. 12/282,766, filed on Oct. 14, 2008.
(60) Provisional application No. 60/783,187, filed on Mar. 16, 2006.

(51) Int. Cl. C07K 2/00

(52) U.S. Cl. 530/345

(57) ABSTRACT

A method is disclosed for the syntheses of thiol-containing radiopharmaceuticals without the need for purification starting from chelators containing disulfide bonds. This is done by providing a method that reduces disulfide bonds on a precursor molecule or a precursor compound in the presence of phosphine compounds, thus freeing thiols for metal complexation.

### FIELD OF INVENTION

The present invention relates to formulations for radiopharmaceuticals comprising radionuclide chelators. It also relates to novel methods and formulations for the preparation of thiol-containing metal complexes by using phosphine reduction of a disulfide-containing ligand to form an active chelating thiolate-bearing ligand.

### BACKGROUND OF THE INVENTION

Targeted radiopharmaceuticals are designed to deliver a radioisotope to a specific target in a body for imaging or therapeutic purposes. Targeting molecules include monoclonal and polyclonal antibodies and fragments, proteins, peptides and non-peptides. Targeting molecules have been radiolabeled with metal radionuclides. Typical metals used for diagnostic imaging include ^{99m}Tc, ⁶⁴Cu, ⁶⁷Cu, ⁹⁷Ru, ¹⁰⁹Pd, ¹⁹⁸Au, ⁶⁷Ga, ⁶⁸Ga, ⁹⁴Tc, ^{94m}Tc and ¹¹¹In while typical metal radionuclides used for radiotherapy include ¹⁸⁶Re, ¹⁸⁸Re, ¹¹¹In, ¹⁶⁶Ho, ¹⁰⁵Rh, ¹⁴⁹Pm, ¹⁵³Sm, ¹⁷⁷Lu, ⁹⁰Y, ²⁰³Pb, ²¹²Pb and ²¹²Bi. Metal radionuclides can be linked to a targeting molecule mainly through two different approaches.

The first approach employs direct labeling by exploiting, for example, the presence of thiolate groups of cysteine side chains, usually generated by reduction of a disulfide bond present in peptides, proteins or antibodies. This approach is simple because it does not require synthetic modification of the biological molecule, but can lead to the formation of a conjugate with an unpredictable structure, sometimes with limited in vivo stability.

The second approach employs labeling of a chelator attached through a linker to a targeting molecule. This approach can be further divided in two main categories: (1) labeling of a metal radionuclide with a chelator that is previously linked to a targeting molecule; and (2) labeling of a metal radionuclide with a chelator that contains a functional group that can be subsequently reacted with a targeting group.

In both of these latter two cases, the structure of the Conjugate is predictable and the stability of the metal complex can be optimized using different donor atoms and chelator frameworks. Moreover the pharmacokinetics of the radiopharmaceutical can be fine tuned by modifying the linker between the chelator and the targeting molecule.

Targeted radiopharmaceuticals based on ⁹⁴Tc, ^{94m}Tc, ^{99m}Tc, ¹⁸⁸Re, or ¹⁸⁶Re can be labeled by reaction with a reducing agent that reduces the metal from an oxidized state to a reduced state that can coordinate with the desired chelator. Useful reducing agents include, for example, stannous chloride, stannous pyrophosphate, stannous fluoride, stannous tartrate, stannous glucoheptonate, stannous DTPA, sodium or other salts of borohydride and the like.

Multidentate ligands that contain at least one thiol group are known to form unusually stable coordination complexes of technetium and rhenium. Several such ligand types are known. Investigations have shown that amide thiolate (N₂S₂, N₃S) cheating agents form well defined ^{99m}Tc complexes of high stability (Davison et al. Inorg. Chem., 1981, 20, 1629-1632; Kasina et al. J. Med. Chem. 1986, 29, 1933-1940).

For example, the N₂S₂ and N₃S amide thiol containing chelates such as DADS and MAG3 (disclosed by U.S. Pat. No. 4,980,147 and by Davison et al.) form anionic technetium complexes.

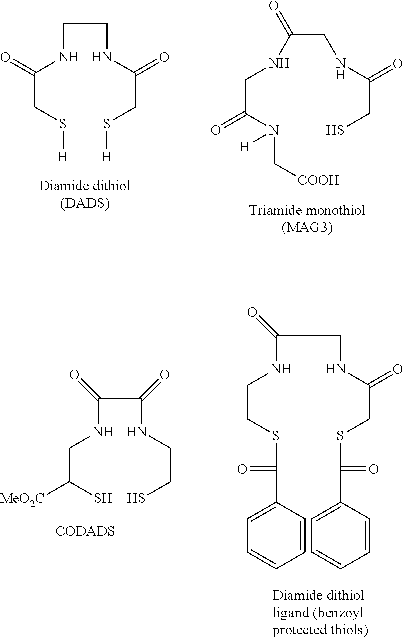

The targeted N₂S₂ peptide known as P483 contains the Cys-Gly-Cys tripeptide chelating unit and has been disclosed for use in inflammation imaging (WO 94/28942 A1).

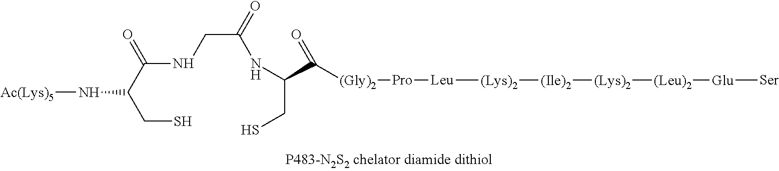

The N₂S₂ diamine dithiol ligand known as BAT was disclosed by Kung (EP 0 200 21) and forms a neutral lipophilic technetium complex.

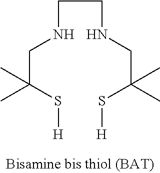

Monoamine bisamide mono thiol ligands containing the N₃S donor set have been disclosed by Goodbody and Pollack in Peptide-chelator conjugates for diagnostic imaging (U.S. Pat. Nos. 5,662,885; 5,780,006 and 5,976,495). One example is shown

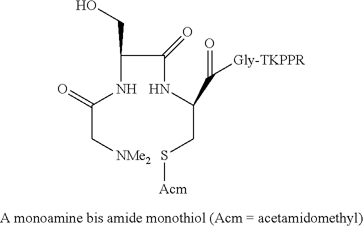

Archer et al. (J. Chem. Soc., Dalton Trans: Inorganic Chemistry, 1997, (8), 1403-1410) have described tetradentate ligands for technetium and rhenium with N₂S₂ and N_{S}3 donor sets. Two such compounds are shown below.

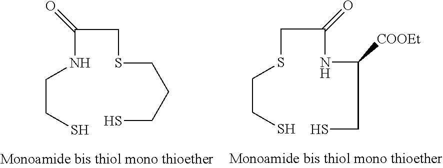

Other peptide based thiol-containing ligands for technetium have been described in a review on this subject (Liu et al. Chem. Rev., 1999, 99(9), 2235-2268).

Peptide dictators based on Pic-Ser-Cys are disclosed by Pollak et al. (WO 95/17419).

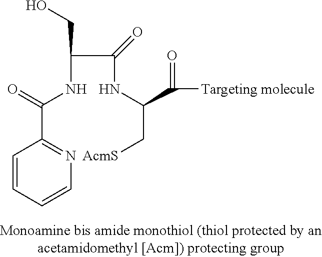

Rey et al. (Appl. Radiat. Isot. 2001, 54(3), 429-434) have disclosed Tc complexes prepared by the reaction of a tetradentate bis thiol-containing ligand and a monothiol.

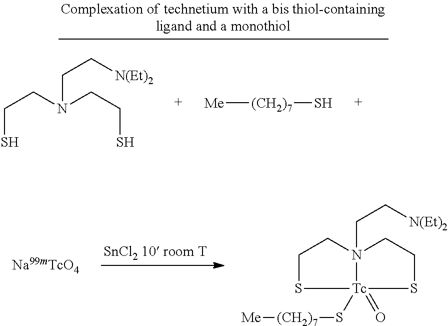

All of these thiol-containing ligands and others can undergo reaction oxygen to form disulfide bonds. Such disulfide bond formation can be either intramolecular or intermolecular.

The tendency of thiols to oxidize to disulfides makes the manufacture and formulation of products based on thiol-containing ligands challenging, as oxidation of thiols to disulfides reduces the purity of the ligand and lowers the amount of thiol-containing ligand available for reaction with a radiometal such as technetium or rhenium, as these metals are not known to form stable chelates with disulfides. This problem becomes worse at low ligand concentrations.

In addition, the chelator-biological molecule conjugates used to make targeted radiopharmaceuticals can often have biological effects that are similar to those of the natural ligands that bind to the target receptor. For example the targeting group on the ligand used to prepare ^{99m}Tc Compound 1 (also known as ^{99m}TcRP-527) causes similar biological effects to those of natural ligands such as bombesin and gastrin releasing peptide (GRP), compounds that bind to GRP receptors. Bombesin is a growth factor for a number of human cancer cell lines, including small cell lung cancer (SCLC), breast and prostate cancer, and has potent biological effects including effects on blood pressure, tachyphylaxis, stimulation of gastric acid and pancreatic enzyme secretion, effects on peristaltic activity, satiety and effects on thermoregulation. Because of these potent effects, it would be highly advantageous to have a radiolabeling method that significantly reduces the amount of chelator needed to produce the desired product in high yield.

The oxidative instability of the sulfhydryl groups can compromise the syntheses of N₂S₂ and N₃S chelating agents in a highly pure form and is an obstacle to prolonged storage. For these reasons the thiols in these molecules are usually protected by a suitable protecting group. Protecting groups for sulfhydryl groups include benzoyl, benzyl, benzamidomethyl, acetamidomethyl (Acm), acetyl, 1-ethoxyethyl, tetrahydropyranyl, p-methoxybenzyl, diphenylmethyl and triphenylmethyl (Davison et al. Eur. Pat. Appl. EP 135160). These protecting groups arc subsequently removed either prior to or during the labeling reaction, yielding the free thiol after removal. Investigations have demonstrated that it is possible to remove these protecting groups with reducing agents, but at high temperature and using relatively large concentrations of the chelator (Davison et al. Eur. Pat, Appl. EP 135160).

Sometimes the procedure for labeling of N₂S₂, N₃S dictators requires purification steps that render the process infeasible for commercial production (Okarvi et al. J. Labelled Compd. Radiopharm., 1997, Vol. XXXIX, No. 10; Liu et al. Bioconjugate Chem, 1996, 7 (2), 196-202). The radiopharmaceutical ^{99m}TcRP-527 with the structure shown below is an example of a compound having the problems previously described (potency of peptide, harsh labeling conditions, and need for HPLC purification).

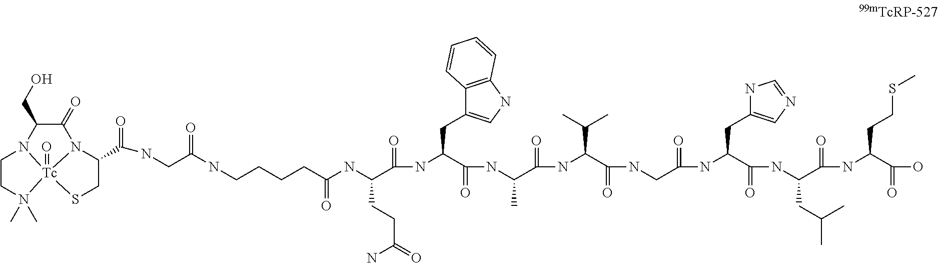

The N₃S chelator N-Me₂-Gly-Ser-Cys-Acm used to prepare ^{99m}TcRP-527 is linked to the N-terminus of an octapeptide targeting molecule, Gln-Trp-Ala-Val-Gly-His-Leu-Met-NH₂, via a Gly-aminovaleroyl linker. Prior to radiolabeling, the thiol present in the N₃S chelator is protected with an acetamido (Acm) group that is lost during the labeling reaction with ^{99m}TcO₄⁻, thus allowing the thiol group to coordinate to ^{99m}Tc. Both the RP-527 ligand and ^{99m}TcRP-527 bind to the Gastrin-Releasing Peptide Receptor (GRP-R), which is over expressed in several types of cancer, including prostate, breast and small cell lung cancer.

Clinical studies were performed with this compound by Van de Wiele et al (Eur. J. Nucl. Med., 2000, Vol 27 (11), 1694). In these studies, this compound was prepared using the following 4-vial kit. To each of 2 vials, each containing 100 μg of ligand was added 0.1 mL of stannous chloride (2 mM), 0.1 mL of sodium gluconate (60 mmol), 1850-2035 MBq (50-55 mCi) of ^{99m}TcO₄⁻ in 0.3 mL of 0.9% sodium chloride, and 0.5 mL of saline (0.9% sodium chloride). After 35 min. in a boiling water bath, the pooled reaction mixtures were injected in an HPLC system and purified in order to separate labeled from unlabeled peptide, followed by terminal sterilization. The overall yield from this radiosynthesis was ˜30%, with a radiochemical purity after purification of >90%. The purified compound could be stored at 4° C. for only up to 2 hours. This procedure, although valuable for early clinical studies, is unacceptable for commercial purposes.

It would be useful to have a procedure to prepare this and other thiol-containing; targeted radiopharmaceuticals that uses lower amounts of ligands, and without the need for HPLC purification.

### SUMMARY OF THE INVENTION

The present invention provides a molecule comprised of at least two linked compounds, wherein: (a) prior to linking, each compound comprises a metal chelating group containing at least one thiol group necessary for metal chelation; (b) each compound is covalently joined to another compound by disulfide bonds between the thiol groups, thus linking two chelating groups together; and (c) each compound has a structure of the formula. X-Y-B wherein X is the metal chelating group, Y is a spacer group or covalent bond and B is a targeting group.

The present invention also provides a compound comprising a chelating group attached to a targeting group wherein: (a) said compound has a structure of the formula X-Y-B wherein X is a metal chelating group, Y is a spacer group or covalent bond and B is a targeting group; and (b) said chelating group has a thiol group necessary for metal chelation and forms a disulfide bond with another thiol group on any part of the compound.

The present invention further provides a method of complexing a metal to a chelating group comprising at least one thiol, said method comprising the following steps: (i) providing a disulfide-containing precursor compound or precursor molecule, wherein said thiol is bound to a second thiol forming an intramolecular disulfide bond in the precursor compound or an intermolecular disulfide bond in the precursor molecule; and

(ii) reducing said disulfide bond by treating said precursor compound or precursor molecule with a phosphine compound in the presence of said metal, thereby forming said complex.

The present invention also provides a method of complexing a Metal to a thiol group, said method comprising the following steps: (i) providing a disulfide-containing precursor compound, wherein said thiol is bound to a second thiol forming an intermolecular disulfide bond; and (ii) reducing said disulfide bond by treating said precursor compound with a phosphine compound in the presence of said metal, thereby forming said complex.

The present invention further provides a kit for the preparation of a radiopharmaceutical agent, said kit comprising a molecule comprised of at least two linked compounds, wherein: (a) prior to linking, each compound comprises a metal chelating group containing at least one thiol group necessary for metal dictation; (b) each compound is covalently joined to another compound by disulfide bonds between the thiol groups linking two chelating groups together; and (c) each compound has a structure of the formula X-Y-B wherein X is the metal chelating group, Y is a spacer group or covalent bond and B is a targeting group.

The present invention also provides a kit for the preparation of a radiopharmaceutical agent, said kit comprising a compound comprised of a chelating group attached to a targeting group wherein: (a) said compound has a structure of the formula X-Y-B wherein X is a metal chelating group, Y is a spacer group or covalent bond and B is a targeting group; and (b) said chelating group has a thiol group necessary for metal chelation and forms a disulfide bond with another thiol group on any part of the compound.

The present invention further provides a method of preparing a molecule comprised of two compounds, wherein each compound has a structure of the formula X-Y-B, X is a metal chelating group containing at least one thiol group necessary for metal chelation, Y is a spacer group or covalent bond; and B is a targeting group, said method comprising covalently joining said two compounds by at least one disulfide bond between the thiol groups, thereby linking the two chelating groups together and preparing said molecule.

The present invention also provides a method of preparing the compound of the present invention comprising:

(1) providing a substrate compound, wherein said substrate compound: (a) comprises a chelating group attached to a targeting group; (b) has a structure of the formula. X-Y-B wherein X is a metal chelating group, Y is a spacer group or covalent bond and B is a targeting group; and (c) has at least two thiol groups, at least one of which is in said chelating group and is necessary for metal chelation; and

(2) forming a disulfide bond between the thiol group in the chelating group and another thiol group on any part of the substrate compound.

### DETAILED DESCRIPTION OF THE INVENTION

In the following description, various aspects of the present invention will be further elaborated. For purposes of explanation, specific configurations and details are set forth in order to provide a thorough understanding of the present invention. However, it will also be apparent to one skilled in the art that the present invention may be practiced without the specific details. Furthermore, well known features may be omitted or simplified in order not to obscure the present invention.

The present invention provides a molecule comprised of at least two linked compounds, wherein: (a) prior to linking, each compound comprises a metal chelating group containing at least one thiol group necessary for metal chelation; (b) each compound is covalently joined to another compound by disulfide bonds between the thiol groups, thus linking two chelating groups together; and (c) each compound has a structure of the formula X-Y-B wherein X is the metal chelating group, Y is a spacer group or covalent bond and B is a targeting group.

In a preferred embodiment, each compound comprises a metal chelating group X which is a monoamine bis amide monothiol chelator attached to a targeting group B via a spacer group or covalent bond Y. The two compounds are covalently joined by disulfide bonds between the thiol groups. The spacer and targeting group in each compound may be the same or different. In a particularly preferred embodiment each compound has the structure:

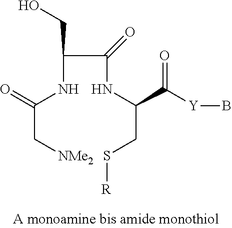

where R is H or a thiol protecting group. The spacer and targeting group in each compound may be the same or different. The compounds are covalently joined by disulfide bonds between the thiol groups to form a molecule of the structure:

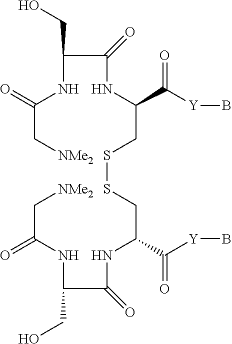

The present invention also provides a compound comprising a chelating group attached to a targeting group wherein: (a) said compound has a structure of the formula X-Y-B wherein X is a metal chelating group, Y is a spacer group or covalent bond and B is a targeting group; and (h) said chelating group has a thiol group necessary for metal chelation and forms a disulfide bond with another thiol group on any part of the compound. In a preferred embodiment, the compound comprises a metal chelating group X which is a his amide bis thiol chelator or a his amine bis thiol chelator attached to a targeting group B via a spacer group or covalent bond Y. A thiol in the chelating group X forms a disulfide bond with another thiol group in the compound. In a preferred embodiment the second thiol group is also in the chelating group X. In a particularly preferred embodiment the compound has the structure:

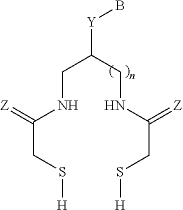

wherein n is 0 or 1 and both Z's are O (bis amide his thiol) or absent (his amine his thiol). A thiol in the chelating group X forms a disulfide bond with another thiol group in the compound, forming the structure. In a preferred embodiment the second thiol group is also in the chelating group X:

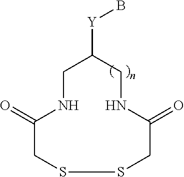

The present invention allows for the syntheses of thiol-containing radiopharmaceuticals without the need for purification, starting from chelators containing disulfide bonds. This is done by providing a method that reduces disulfide bonds on a precursor molecule or a precursor compound in the presence of phosphine compounds, thus freeing thiols for metal complexation.

Specifically, a precursor molecule is comprised of at least two linked compounds, wherein: (a) prior to linkage, each compound comprises a metal chelating group containing at least one thiol group necessary for metal chelation; (b) each compound is covalently joined to another compound by disulfide bonds between the thiol groups linking two chelating groups together; and (c) each compound has a structure of the formula X-Y-B wherein X is the metal chelating group, Y is a spacer group or covalent bond and B is a targeting group.

Preferably, each compound of the precursor molecule comprises a metal chelating group X which is a monoamine his amide monothiol chelator attached to a targeting group B via a spacer group or covalent bond Y. The two compounds arc covatently joined by disulfide bonds between the thiol groups. The spacer and targeting group in each compound may be the same or different. In a particularly preferred embodiment each compound has the structure:

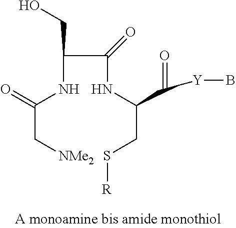

where R is H or a thiol protecting group. The spacer and targeting group in each compound may be the same or different. The compounds are covalently joined by disulfide bonds between the thiol groups to form a homodimer precursor molecule of the structure:

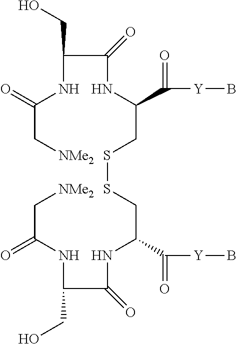

In another preferred embodiment, the precursor molecule is a homodimer of the structure:

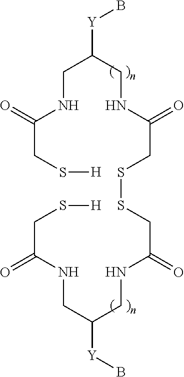

where n=0 or 1. Even more preferably, the precursor molecule of the present invention is a homodimer compound 2 or compound 15 shown below (5Ava=5 aminovaleroyl):

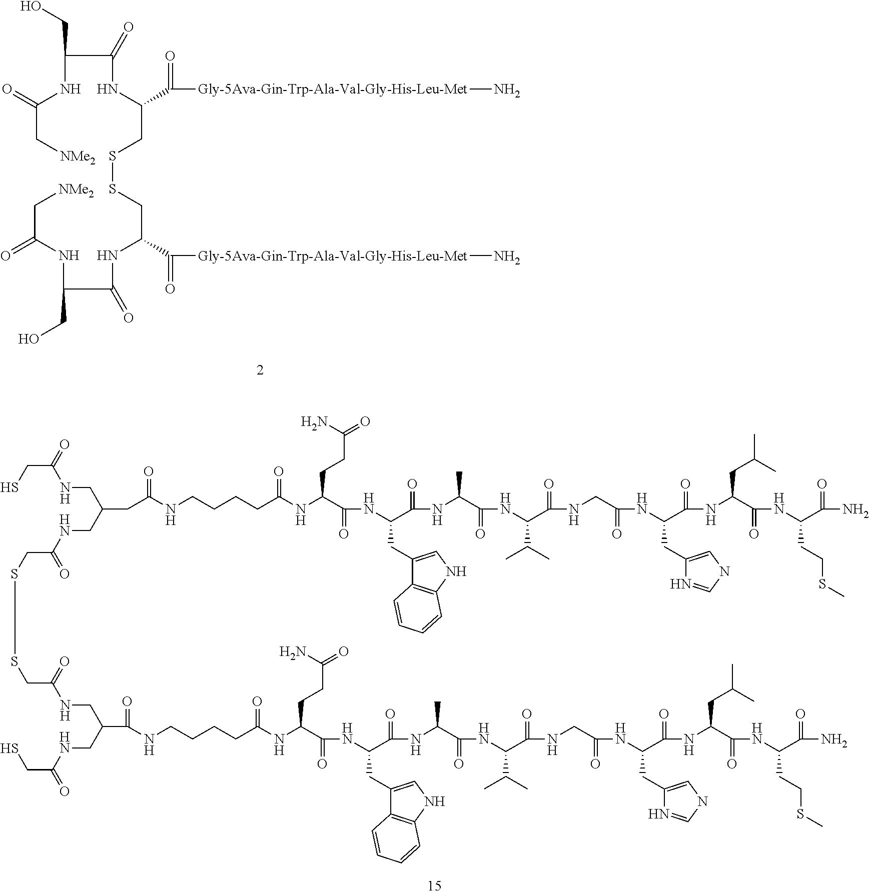

A precursor compound may also comprise a chelating group attached to a targeting group wherein: (a) said compound has a structure of the formula X-Y-B wherein X is a metal chelating group, Y is a spacer group or covalent bond and B is a targeting group; and (b) said chelating group has a thiol group necessary for metal chelation and forms a disulfide bond with another thiol group on any part of the compound. Preferably, the other thiol group is also in the chelating group X and thus t disulfide bond is formed between two thiol groups within the chelating group.

In a preferred embodiment, the precursor compound comprises a metal chelating group X which is a his amide his thiol chelator or a his amine his thiol chelator attached to a targeting group B via a spacer group or covalent bond Y. A thiol in the chelating group X forms a disulfide bond with another thiol group in the compound. In a preferred embodiment the second thiol group is also in the chelating group X. In a particularly preferred embodiment, the chelating group has the structure set forth below and is attached to Y-B as indicated:

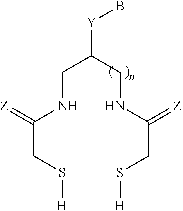

wherein n is 0 or 1 and both Z's are O (bis amide his thiol) or absent (his amine his thiol). A thiol in the chelating group X forms a disulfide bond with another thiol group in the compound, forming a precursor compound of the structure:

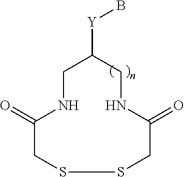

In a preferred embodiment the second thiol group is also in the chelating group X, as indicated in the structure above.

Most preferably, in this embodiment, the precursor compound of the present invention is compound 9 or compound 14 shown below:

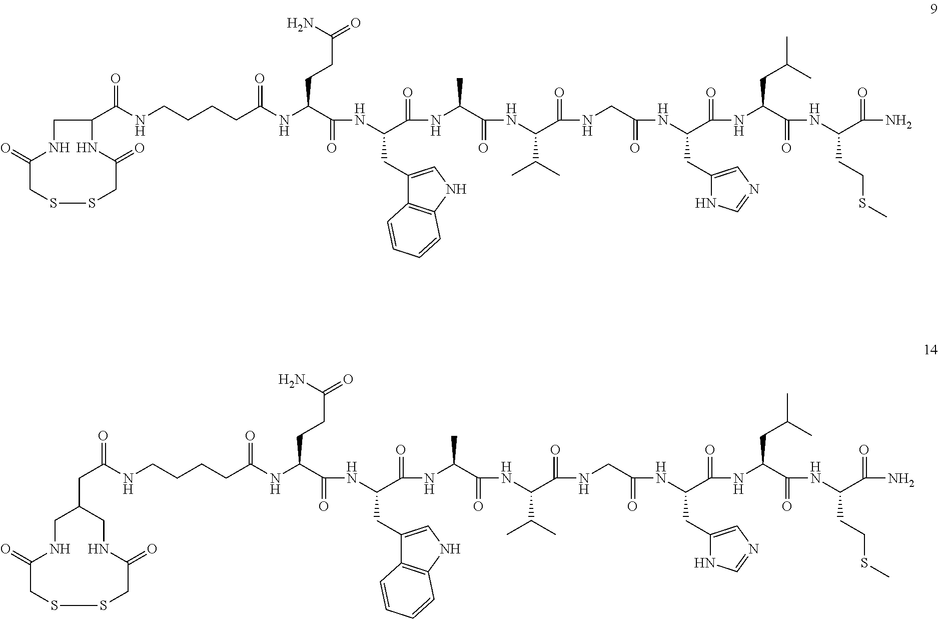

The present invention can be applied to a wide variety of radiopharmaceuticals that are formed by reduction of a radioactive metal using an excess of reducing agent in the presence of a chelating or complexing ligand that has one or more protected thiols for coordination to the radioactive metal. The present invention also allows for the deprotection of sulfhydryl groups by mean of phosphine reduction of disulfide bonds without reducing the metal center to phosphine-containing lower oxidation states that do not have the same desirable characteristics as the desired product.

### Metal Chelating Group X

The term “metal chelating group” refers to a molecule or a fragment thereof that forms a complex with a metal atom, wherein said complex is stable under physiological conditions. That is, the metal will remain complexed to the chelator backbone in vivo. More particularly, a metal chelator is a molecule that complexes to a radionuclide metal to form a metal complex that is stable under physiological conditions and which also may be conjugated with a targeting group through linker Y. The metal chelator X may be any of the thiol-containing metal chelators known in the art for complexing a medically useful metal ion or radionuclide. The metal chelator may or may not be complexed with a metal radionuclide.

The metal chelators of the invention may include, for example, linear, macrocyclic, terpyridine, and N₃S, or N₇S₂ chelators (see also, U.S. Pat. No. 5,367,080, U.S. Pat. No. 5,364,613, U.S. Pat. No. 5,021,556, U.S. Pat. No. 5,075,099, U.S. Pat. No. 5,886,142, the disclosures of which are incorporated by reference herein in their entirety), and other chelators known in the art including, but not limited to bisamino bisthiol (BAT) chelators (see also U.S. Pat. No. 5,720,934). Certain preferred N₃S chelators are described in PCT/CA94/00395, PCT/CA94/00479, PCT/CA95/00249 and in U.S. Pat. Nos. 5,662,885; 5,976,495; and 5,780,006, the disclosures of which are incorporated by reference herein in their entirety. The chelator may also include derivatives of the chelating ligand mercapto-acctyl-glycyl-glycyl-glycine (MAG3), which contains an N₃S, and N₂S₂ systems such as MAMA (monoamidemonoaminedithiols), DADS (N₂S diaminedithiols), CODADS and the like. These ligand systems and a variety of others are described in Liu and Edwards (Chem. Rev. 1999, 99, 2235-2268) and references therein, the disclosures of which are incorporated by reference herein in their entirety.

The monoamine bis amide monothiol chelators have the general formula

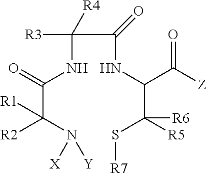

when
X is a linear or branched, saturated or Unsaturated C₁₋₄ alkyl chain that is optionally interrupted by one or two heteroatoms selected from N, O, and S; and optionally substituted by at least one group selected from halogen, hydroxyl, amino, carboxyl, C₁₋₄ alkyl, aryl and C(O)Z;
Y is H or a substituent defined by X;
X and Y may together form a 5- to 8-membered saturated or unsaturated heterocyclic ring optionally substituted by at least one group selected from halogen, hydroxyl, amino, carboxyl, oxo, C₁₋₄ alkyl, aryl and C(O)Z;
R¹ through R⁴ are selected independently from H; carboxyl; substituted with a group selected from hydroxyl, amino, sulfhydryl, halogen, carboxyl, C₁₋₄ alkoxycarbonyl and aminocarbonyl; an alpha carbon side chain of a D- or 1-amino acid other than proline; and C(O)Z;
R⁵ and R⁶ are selected independently from H; carboxyl; C₁₋₄ alkyl; C₁₋₄ alkyl substituted by hydroxyl, carboxyl or amino; and C(O)Z;
R⁷ is selected from H and a sulfur protecting group; and
Z is selected from hydroxyl and a targeting molecule. One skilled in the art would understand that Y (the spacer group or covalent bond) and B (the targeting group) of the present invention may be attached, for example, at those positions where Z (or C(O)Z) may be attached in the general formula.

In a preferred embodiment, the targeted chelators conform to the above formula in which:

R¹ through R⁴ are selected independently from H; and a hydroxy-substituted C₁₋₄ alkyl group such as hydroxymethyl and 1-hydroxyethyl;

R⁵ and R⁶ are select independently from H and (C₁₋₄ alkyl, and are preferably both H,

R⁷ is a hydrogen atom or a sulfur protecting group and is most preferably a hydrogen atom.

Particularly preferred chelators include N,N-dimethylGlycine-Ser-Cys and N,N-dimethylGlycine-Thr-Cys. The metal chelators of the invention may also include, for example, the monoamine, his amide monothiol N₃S chelators described in. EP 0804252, incorporated by reference herein in its entirety, wherein the metal dictator is selected from

(i) a group having the formula:

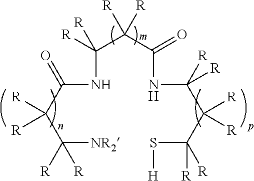

and

(ii) a group having the formula:

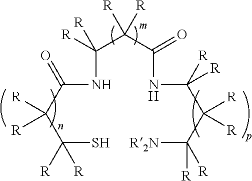

wherein:
n, m and p are each independently 0 or 1;
each R′ is independently H, lower alkyl, hydroxyalkyl (C₂-C₄), or alkoxyalkyl (C₂-C₄);
each R is independently H or R″ where R″ is substituted or unsubstituted lower alkyl or phenyl, not comprising a thiol group;
one R or R′ is L, wherein when R′ is L, NR′₂ is an amine; and L is a bivalent linking group linking the chelator to the targeting moiety. One skilled in the art would understand that Y (the spacer group or covalent bond) and B (the targeting group) of the present invention may be attached, for example, at those positions where L may be attached in the general formulae above.

Preferred chelators of formula II have the structure

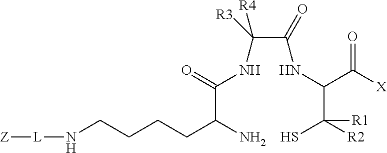

where in R¹ and R² are independently H, lower alkyl, hydroxyalkyl (C₂-C₄ or alkoxyalkyl (C₂-C₄):
R³ and R⁴ are independently H, substituted or unsubstituted lower alkyl or phenyl not comprising a thiol group, X is NH₂, NR¹R² or NR¹—Y, where Y is an amino acid, an amino acid amide, or a peptide of from 2 to about 20 amino acids; L is a bivalent linking moiety; and Z is a targeting moiety. One skilled in the art would understand that Y (the spacer group or covalent bond) and B (the targeting group) of the present invention may be attached, for example, where L-Z is attached in the above structure.

The chelator may also include derivatives of the chelating ligand mercapto-acetyl-glycyl-glycyl-glycine (MAG3), which contains an N₃S, and N₂S₂ systems such as MAMA (monoamidemonoaminedithiols), DADS (N₂S diaminedithiols), CODADS and the like. These ligand systems and a variety of others are described in Liu and Edwards, Chem Rev, 1999, 99, 2235-2268 and references therein, the disclosures of which are incorporated by reference herein in their entirety.

Examples of preferred chelators include, but are not limited to BAT, DADS, MAG3, CODADS, N₃S, N₂S₂, NS₃ and derivatives thereof. N₃S monoamine his amide monothiol dictators are particularly preferred chelators, with chelators within the following formula being especially preferred:

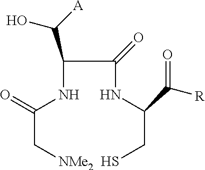

where A is H or CH₃ and R is OH or NH₂.
Similarly N₂S₂ chelators are also preferred, with chelators of the following formula being especially preferred:

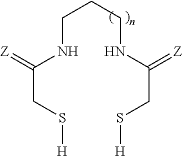

wherein n is 0 or 1 (so 5 or 6-membered ring) and both Z's are O (bis amide bis thiol) or absent (bis amine bis thiol).

Preferred metal radionuclides for scintigraphy or radiotherapy include ⁹⁴Tc, ^{94m}Tc, ^{99m}Tc, ⁵¹Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho, ¹⁶⁵Dy, ¹⁶⁶Dy, ⁶²Cu, ⁶⁴Cu, ⁹⁷Ru, ¹⁰³Ru, ¹⁸⁶Re, ¹⁸⁸Re, ²⁰³Pb, ²¹¹Bi, ²¹²Bi, ²¹³Bi, ²¹⁴Bi, ¹⁰⁵Rh, ¹⁰⁹Pd, ^{117m}Sn, ¹⁴⁹Pm, ¹⁶¹Tb, ¹⁷⁷Lu, ¹⁹⁸Au and ¹⁹⁹Au and oxides or nitrides thereof. The choice of metal will be determined based on the desired therapeutic or diagnostic application. For example, for diagnostic purposes (e.g., to diagnose the presence of receptors or to monitor therapeutic progress in primary tumors and metastases), the preferred radionuclides include ⁶⁴Cu, ⁶⁷Ga, ⁶⁸Ga, ^{99m}Tc, and ¹¹¹In, with ^{99m}Tc and ¹¹¹In being especially preferred. For therapeutic purposes (e.g., to provide radiotherapy for primary tumors and metastasis related to cancers of the prostate, breast, lung, etc.), the preferred radionuclides include ⁶⁴Cu, ⁹⁰Y, ¹⁵⁰Rh, ¹¹¹In, ^{117m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, ¹⁸⁶Re, ¹³³Re, and ¹⁹⁹Au, with ¹⁸⁶Re and ¹⁸⁸Re being particularly preferred. ^{99m}Tc is particularly useful and is preferred for diagnostic applications because of its low cost, availability, imaging properties, and high specific activity. The nuclear and radioactive properties of ^{99m}Tc make this isotope an ideal scintigraphic imaging agent. This isotope has a single photon energy of 140 keV and a radioactive half-life of about 6 hours, and is readily available from a ⁹⁹Mo-^{99m}Tc generator.

### Spacer Group Y

Spacer group Y may be a covalent bond or a spacer or linking group. In a preferred embodiment of the present invention, the spacer group Y is selected from the group consisting of a covalent bond, at least one amino acid residue, a hydrocarbon chain and a combination thereof. More preferably, the spacer group Y is selected from the group consisting of glycine, β-alanine, gamma-aminobutanoic acid, 5-aminovaleric acid (5-Ava), 6-aminohexanoic acid, 7-aminoheptanoic acid, 8-aminooetanoic acid (8-Aoc), 9-aminononanoic acid, 10-aminodecanoic acid and 11-aminoundecanoic acid (11-Aun). Other spacer groups may also include a pure peptide linking group consisting of a series of amino acids (e.g., diglycine, triglycine, Gly-Ser-Gly, etc.). Preferred spacer groups Y are is Gly-Ser-Gly and Gly-(5-Ava).

Other spacer groups can also include a hydrocarbon chain [i.e., R₁—(CH₂)—R₂] wherein n is 0-10, preferably n=3 to 9, R₁ is a group (e.g., H₂N—, HS—, —COOH) that can be used as a site for covalently linking the metal chelator; and R₂ is a group that is used for covalent coupling to the N-terminal NH₂-group of a given targeting peptide (e.g., R₂ is an activated COOH group) or other diagnostic or therapeutic moiety. Several chemical methods for conjugating chelators to targeting groups have been well described in the literature (Wilbur, 1992; Parker, 1990; Hermanson, 1996; Frizberg et al., 1995). These methods include the formation of acid anhydrides, aldehydes, arylisothiocyanates, activated esters, or N-hydroxysuccinimides [Wilbur, 1992; Parker, 1990; Hermanson, 1996; Frizberg et al., 1995].

Other spacer groups may be formed from spacer precursors (SP) having electrophiles or nucleophiles as set forth below:
SP1: a spacer precursor having on at least two locations of the linker the sane electrophile E1 or the same nucleophile Nu1;
SP2: a spacer precursor having an electrophile E1 and on another location of the linker a different electrophile E2;
SP3: a spacer precursor having a nucleophile Nut and on another location of the linker a different nucleophile Nu2; or
SP4: a spacer precursor having one end functionalized with an electrophile E1 and the other with a nucleophile Nu1.

The preferred nucleophiles Nu1/Nu2 include —OH, —NH, —NR, —SH, —HN—NH₂, —RN—NH₂, and —RN—NHR′, in which R′ and R are independently selected from the definitions for R given above, but for R′ is not H.

The preferred electrophiles E1/E2 include —COOH, —CH═O (aldehyde), —CR═OR′ (ketone), —RN—C═S, —RN—C═O, —S—S-2-pyridyl, —SO₂—Y, —CH₂C(═O)Y, and

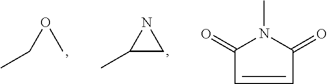

wherein Y can be selected from the following groups:

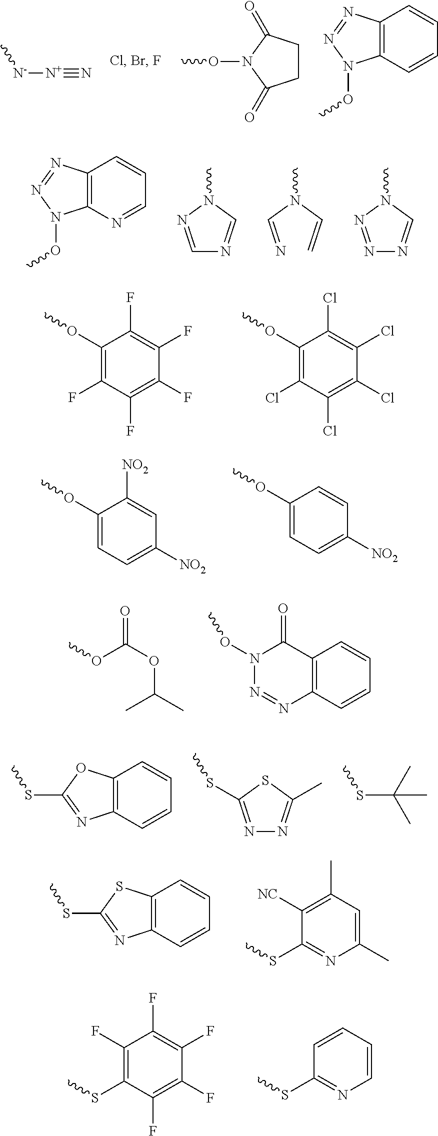

The spacer group Y may also contain at least one substituted bile acid. Bile acids are found in bile (a secretion of the liver) and are steroids having a hydroxyl group and a five carbon atom side chain terminating in a carboxyl group. In substituted bite acids, at least one atom such as a hydrogen atom of the bile acid is substituted with another atom, molecule or chemical group. For example, substituted bile acids include those having a 3-amino, 24-carboxyl function optionally substituted at positions 7 and 12 with hydrogen, hydroxyl or keto functionality. Other substituted bile acids useful as linkers in the present invention include substituted cholic acids and derivatives thereof. Specific substituted cholic acid derivatives include: (3β,5β)-3-aminocholan-24-oic acid; (3β,5β,12α)-3-amino-12-hydroxycholan-24-oic acid; (3β,5β,7α,12α)-3-amino-7,12-dihydroxycholan-24-oic acid; Lys-(3,6,9)-trioxaundecane-1,11-dicarbonyl-3,7-dideoxy-3-aminocholic acid); (3β,5β,7α)-3-amino-7-hydroxy-12-oxocholan-24-oic acid; an (3β,5β,7α)-3-amino-7-hydroxycholan-24-oic acid.

In another embodiment, the spacer group Y contains at least one non-alpha amino acid. Preferred non-alpha amino acids include:
8-amino-3,6-dioxaoctanoic acid;
N-4-aminoethyl-N-1-acetic acid; and
polyethylene glycol derivatives having the formula NH₂—(CH₂CH₂O)n-CH₂CO₂H or NH₂—(CH₂CH₂O)n-CH₂CH₂CO₂H where n=2 to 100

In a more preferred embodiment, the spacer group Y contains at least one non-alpha amino acid with a cyclic group. Non-alpha amino acids with a cyclic group include substituted phenyl, biphenyl, cyclohexyl or other amine and carboxyl containing cyclic aliphatic or heterocyclic moieties. Examples of such include:

4-aminobenzoic acid (hereinafter referred to as
“Abz4 in the specification”)
3-aminobenzoic acid
4-aminomethyl benzoic acid
8-aminooctanoic acid
trans-4-aminomethylcyclohexane carboxylic acid
4-(2-aminoethoxy)benzoic acid
isonipecotic acid
2-aminomethylbenzoic acid
4-amino-3-nitrobenzoic acid
4-(3-carboxymethyl-2-keto-1-benzimidazolyl-piperidine
6-(piperazin-1-yl)-4-(3H)-quinazolinone-3-acetic acid
(2S,5S)-5-amino-1,2,4,5,6,7-hexahydro-
azepino[3,21-hi]indole-4-one-2-carboxylic acid
(4S,7R)-4-amino-6-aza-5-oxo-9-thiabicyclo[4.3.0]nonane-
7-carboxylic acid
3-carboxymethyl-1-phenyl-1,3,8-triazaspiro[4.5]decan-4-one
N1-piperazincacetic acid
N-4-aminoethyl-N-1-piperazineacetic acid
(3S)-3-amino-1-carboxymethylcaprolactam
(2S,6S,9)-6-amino-2-carboxymethyl-
3,8-diazabicyclo-[4,3,0]nonane-1,4-dione
3-amino-3-deoxycholic acid
4-hydroxybenzoic acid
4-aminophenylacetic acid
3-hydroxy-4-aminobenzoic acid
3-methyl-4-aminobenzoic acid
3-chloro-4-aminobenzoic acid
3-methoxy-4-aminobenzoic acid
6-aminonaphthoic acid
N,N′-Bis(2-aminoethyl)-succinamic acid

These and other spacer groups suitable for the present invention are described in more details in co-pending U.S. Pat. No. 11/165,721 and PCT US04/22115 which are hereby incorporated by reference.

### Targeting Group B

The targeting group for the purpose of the present invention is defined as any molecule that has a binding affinity for a particular site or a specific metabolic function. The targeting group directs the compounds of the invention to the appropriate site, or involves the compounds in a reaction, where the desired diagnostic or therapeutic activity will occur. In an exemplary embodiment, the targeting group may be a monoclonal or polyclonal antibody or fragment thereof, a protein, a peptide or a non-peptide. In a preferred embodiment, the targeting group may be a peptide, equivalent, derivative or analog thereof which functions as a ligand that binds to a particular site. In another exemplary embodiment, the targeting group may be an enzyme, or a molecule that binds to an enzyme. In another exemplary embodiment, the targeting group may be an antibiotic.

In a preferred embodiment, the targeting group is a peptide that binds to a receptor or enzyme of interest. For example, the targeting peptide B may be a peptide hormone such as, for example, luteinizing hormone releasing hormone (LHRH) such as that described in the literature [e.g., Radiometal-Binding Analogues of Luteinizing Hormone Releasing Hormone PCT/US96/08695; PCT/US97/12084 (WO 98/02192)]; insulin; oxytocin; somatostatin; Neuro kinin-1 (NK-1); Vasoactive Intestinal Peptide (VIP) including both linear and cyclic versions as delineated in the literature, [e.g., Comparison of Cyclic and Linear Analogs of Vasoactive Intestinal Peptide. D. R. Bolin, J. M. Cottrell, R. Garippa, N. Rinaldi, R. Senda, B. Simkio, M. O'Donnell. Peptides: Chemistry, Structure and Biology Pravin T. P. Kaumaya, and Roberts S. Hodges (Eds). Mayflower Scientific LTD., 1996, pgs 174-175]; gastrin releasing peptide (GRP); bombesin and other known hormone peptides, as well as analogs and derivatives thereof. More preferably, the targeting peptide is a bombesin agonist binding moiety such as BBN(7-14) and BBN(8-14).

Other useful targeting peptides include analogs of somatostatin such as those described in EP 0 804 252 B1, incorporated herein by reference in its entirety.

Still other useful targeting peptides include Substance P agonists [e.g., G. Bitan et al. Peptides: Chemistry, Structure and Biology, Pravin T. P. Kaumaya, and Roberts S. Hodges (Eds), Mayflower Scientific LTD., 1996, pgs 697-698; and Hidehito at el., j. Biol. Chem. 1992, 267, 16237-16243]; NPY(Y1) [e.g., Soil et al., Eur. J. Biochem. 2001, 268, 2828-283]; Langer et al., Bioconjugate Chem. 2001, 12, 1028-1034; Langer at al., J. Med. Chem. 2001, 44, 1341-13481; oxytocin; endothelin A and endothelin B; bradykinin; Epidural Growth Factor (EGF); [Siemion et al., Peptides 1998, 19, 373-382]; and chotecystokinin (CCK-B) [Eur. J. Nucl Med. 200, 27, 1312-1317].

Literature which gives a general review of targeting peptides, can be found, for example, in the following: The Role of Peptides and Their Receptors as Tumor Markers, Jean-Claude Reubi, Gastrointestinal Hormones in Medicine, Pg 899-939; Peptide Radiopharmaceuticals in Nuclear Medicine, D. Blok, R. I. J. Feitsma, P. Vermeij, E. J. K. Pauwels, Eur. J. Nucl Med. 1999, 26, 1511-1519; and Radiolabeled Peptides and Other Ligands for Receptors Overexpressed in Tumor Cells for Imaging Neoplasms, John G. McAfee, Ronald D. Neumann, Nuclear Medicine and Biology, 1996, 23, 673-676 (somatostatin, VIP, CCK, GRP, Substance P. Galanan, MSH, LHRH, Arginine-vasopressin, endothelia). All of the aforementioned literature in the preceding paragraphs are herein incorporated by reference in their entirety.

Other targeting peptide references include the following: Co-expressed peptide receptors in breast cancer as a molecular basis of in vivo multireceptor tumour targeting. Jean Claude Reubi, Mathias Gugger, Beatrice Waser. Eur. J. Nucl Med. 2002, 29, 855-862, (includes NPY, GRP); Radiometal-Binding Analogues of Luteinizing Hormone Releasing Hormone PCT/US96/08695 (UMW; PCT/US97/12084 (WO 98/02192) (LHRH); PCT/EP90/01169 (radiotherapy of peptides); WO 91701144 (radiotherapy of peptides); and PCT/EP00/01553 (molecules for the treatment and diagnosis of tumours), all of which are herein incorporated by reference in their entirety.

Additionally, analogs of a targeting peptide can be used. These analogs include molecules that target a desired site or receptor with avidity that is greater than or equal to the targeting peptide itself, as well as muteins, retropeptides and retro-inverso-peptides of the targeting peptide. One of ordinary skill will appreciate that these analogs may also contain modifications which include substitutions, and/or deletions and/or additions of one or several amino acids, insofar that these modifications do not negatively alter the biological activity of the peptides described therein. These substitutions may be carried out by replacing one or more amino acids by their synonymous amino acids. Synonymous amino acids within a group are defined as amino acids that have sufficiently similar physicochemical properties to allow substitution between members of a group in order to preserve the biological function of the molecule. Synonymous amino acids as used herein include synthetic derivatives of these amino acids (such as for example the D-forms of amino acids and other synthetic derivatives), and may include those discussed herein. Throughout this application amino acids are abbreviated interchangeably either by their three letter or single letter abbreviations, which are well known to the skilled artisan. Thus, for example, T or Thr stands for threonine, K or Lys stands for lysine, P or Pro stands for proline and R or Arg stands for arginine. For example, one can make the following isosteric and/or conservative amino acid changes in the targeting peptide sequence with the expectation that the resulting peptides would have a similar or improved profile of the properties described above:

Substitution of alkyl-substituted hydrophobic amino acids: Including alanine, leucine, isoleucine, valine, norleucine, S-2-aminobutyric acid, S-cyclohexylalanine or other simple alpha-amino acids substituted by an aliphatic side chain from 1-10 carbons including branched, cyclic and straight chain alkyl, alkenyl or alkynyl substitutions.

Substitution of aromatic-substituted hydrophobic amino acids: Including phenylalanine, tryptophan, tyrosine, biphenylalanine, 1-naphthylalanine, 2-naphthylalanine, 2-benzothienylalanine, 3-benzothienyialanine, histidine, amino, dialkylamino, aza, halogenated (fluoro, chloro, bromo, or iodo) or alkoxy (from C₁-C₄)-substituted forms of the previous listed aromatic amino acids, illustrative examples of which are: 2-, 3-, or 4-aminophenylalanine, 2-, 3-, or 4-chlorophenylalanine, 2-, 3-, or 4-methylphenylalanine. 2-, 3-, or 4-methoxyphenylalanine, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-2-, 3- or 4-biphenylalanine, and 2- or 3-pyridylalanine.

Substitution of amino acids containing basic functions: Including arginine, histidine, ornithine, 2,3-diaminopropionic acid, homoarginine, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma'-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: Including aspartic acid, glutamic acid, homoglutamic acid, tyrosine, alkyl, aryl, aralkyl, and heteroaryl sulfonamides of 2,3-diaminopropionic acid, ornithine or lysine and tetrazole-substituted alkyl amino acids.

Substitution of side chain amide residues: Including asparagine, glutamine, and alkyl or aromatic substituted derivatives of asparagine or glutamine.

Substitution of hydroxyl containing amino acids: Including serine, threonine, homoserine, 2,3-diaminopropionic acid, and alkyl or aromatic substituted derivatives of serine threonine.

It is also understood that the amino acids within each of the categories listed above may be substituted for another of the same group.

Deletions or insertions of amino acids may also be introduced into the defined sequences provided they do not alter the biological functions of said sequences. Preferentially such insertions or deletions should be limited to 1, 2, 3, 4 or 5 amino acids and should not remove or physically disturb or displace amino acids which are critical to the functional conformation, Muteins of the peptides or polypeptides described herein may have a sequence homologous to the sequence disclosed in the present specification in which amino acid substitutions, deletions, or insertions arc present at one or more amino acid positions, Muteins may have a biological activity that is at least 40%, preferably at least 50%, more preferably 60-70%, most preferably 80-90% of the peptides described herein. However, they may also have a biological activity greater than the peptides specifically exemplified, and thus do not necessarily have to be identical to the biological function of the exemplified peptides. Analogs of targeting peptides also include peptidomimetics or pseudopeptides incorporating changes to the amide bonds of the peptide backbone, including thioamides, methylene amines, and E-Olefins. Also peptides based on the structure of a targeting peptide or its peptide analogs with amino acids replaced by N-substituted hydrazine carbonyl compounds (also known as aza amino acids) are included in the term analogs as used herein.

The targeting peptide may be attached to the spacer group via the N or C terminus or via attachment to the epsilon nitrogen of lysine, the gamma nitrogen or ornithine or the second carboxyl group of aspartic or glutamic acid.

The targeting peptide can be prepared by various methods depending upon the selected chelator. The peptide can generally be most conveniently prepared by techniques generally established and known in the art of peptide synthesis, such as the solid-phase peptide synthesis (SPPS) approach. Solid-phase peptide synthesis (SPPS) involves the stepwise addition of amino acid residues to a growing peptide chain that is linked to an insoluble support or matrix, such as polystyrene. The C-terminal residue of the peptide is first anchored to a commercially available support with its amino group protected with an N-protecting agent such as a t-butyloxycarbonyl group (Boc) or a fluorenylmethoxycarbonyl (Fume) group. The amino protecting group is removed with suitable deprotecting agents such as TFA in the case of Boc or piperidine for Fmoc and the next amino acid residue (in N-protected form) is added with a coupling agent such as N,N′-dicyclohexylcarbodiimide (DCC), or N,N′-diisopropylcarbodiimide or 2-(1H-benzotriazol-1-yl)-1,1,3,3-tetramethyluronium hexafluorophosphate (HBTU). Upon formation of a peptide bond, the reagents are washed from the support. After addition of the final residue, the peptide is cleaved from the support with a suitable reagent such as trifluoroacetic acid (TEA) or hydrogen fluoride (HF).

The spacer group may then be coupled to form a conjugate by reacting the free amino group of a selected residue of the targeting peptide with an appropriate functional group of the spacer. The entire construct of chelator, spacer and targeting group discussed above may also be assembled on resin and then cleaved by agency of suitable reagents such as trifluoroacetic acid or HF, as well.

### Methods of Disulfide Bonds

Disulfide bonds are readily formed by air oxidation of a DMSO solution of unprotected thiol groups. This is a preferred method and may be used as set forth in the Examples. Other oxidative methods are also reported in the literature, and known to the skilled artisan including the methods in I, Annis, B. Hargittai, G. Barany, Disulfide bond formation in peptides, Methods Enzymol, 1997, 289, 198-221, Another reagent that has been used for disulfide formation is 4,4′-dithiodipyridine. See D. Cline, C. Thorpe, J. Schneider, General method for facile intramolecular disulfide formation in synthetic peptides. Anat. Biochem. 2004, 168-170. This reagent can be reacted with one sulfide and then a different sulfide can be added to form a hetero-disulfide. Ellman's reagent, 5,5″dithiobis-2-nitrobenzoic acid, has also been used for disulfide formation, See I. Annis, L. Chen, G. Barany, Novel solid-phase reagents for facile formation of intramolecular disulfide bridges in peptides under mild conditions. J. Am. Chem. Soc. 1998, 120, 7226-7238.

### Methods of Complexing Metal to Chelating Group

The present invention further provides a method of complexing a metal to a chelating group comprising at least one thiol, said method comprising the following steps:
(i) providing a disulfide-containing precursor compound or precursor molecule, wherein said thiol is hound to a second thiol forming an intramolecular disulfide bond in the precursor compound or an intermolecular disulfide bond in the precursor molecule; and
(ii) reducing said disulfide bond by treating said precursor compound or precursor molecule with a phosphine compound in the presence of said metal, thereby forming said complex.

The present invention also provides a method of complexing a metal to a thiol group, said method comprising the following steps:

(i) providing a disulfide-containing precursor compound, wherein said thiol is bound to a second thiol forming an intermolecular disulfide bond; and

(ii) reducing said disulfide bond by treating said precursor compound with a phosphine compound in the presence of said metal, thereby funning said complex.

The precursor molecule is preferably comprised of at least two linked compounds, wherein: (a) prior to linking, each compound comprises a metal chelating group containing at least one thiol group necessary for metal chelation; (b) each compound is covalently joined to another compound by disulfide bonds between the thiol groups, thus linking two chelating groups together; and (c) each compound has a structure of the formula X-Y-B wherein X is the metal chelating group, Y is a spacer group or covalent bond and B is a targeting group.

The precursor compound is preferably comprised of a chelating group attached to a targeting group wherein: (a) said compound has a structure of the formula X-Y-B wherein X is a metal chelating group, Y is a spacer group or covalent bond and B is a targeting group; and (b) said chelating group has a thiol group necessary for metal chelation and forms a disulfide bond with another thiol group on any part of the compound.

Preferably, X, Y and B of the precursor molecule and the precursor compound are as described hereinabove.

Also preferably, the phosphine compound is selected from the group consisting trisodium triphenylphosphine-3,3′,3″-trisulfonate (TPPTS), disodium triphenylphosphine-3,3′-disulfonate (TPPDS), sodium triphenylphosphine-3-monosulfonate (TPPMS), Tris(dimethylamino)phosphine, Tris(hydroxymethyl)phosphine and Tris[2-carboxyethyl]phosphine (TCEP), most preferably TCEP shown below:

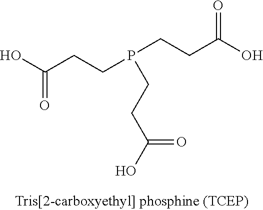

Preferably, the methods of the present invention results in at least 90% yield of said complex. Also preferably, step (ii) is carried out in the presence of a stannous compound, more preferably, in the presence of ethanol and either acetate buffer having a pH of about 5 or sodium bicarbonate buffer having a pH of about 9.

Again preferably, step (ii) of the method of the present invention is carried out at a temperature from about 70° C. to about 100° C., more preferably at about 100° C.

Accordingly, the present invention provides a method for the preparation of radiopharmaceuticals that contain coordinated thiols, wherein the thiol or thiols in the chelating group are protected as disulfide bonds until reaction with water soluble phosphines as described hereinabove.

If the chelating group contains one thiol, the protection of the thiol can be achieved with an intermolecular disulfide bond, thus forming a dimer. The schematic diagram below illustrates the reaction of such a dimer (in this case, a homodimer of an N₃S ligand) with technetium using the phosphine compound TCEP to reduce the disulfide bond.

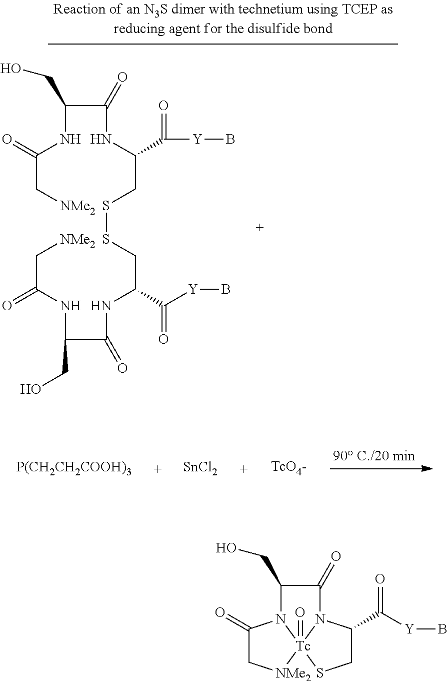

If the chelating group contains two thiols, the protection of the thiols can be achieved with an intramolecular disulfide bond or with an intermolecular disulfide bond with the formation of a dimer. The diagram below illustrates the reaction of technetium with an N₂S₂ metal chelator containing an intramolecular disulfide bond using the phosphine compound TCEP to reduce this disulfide bond.

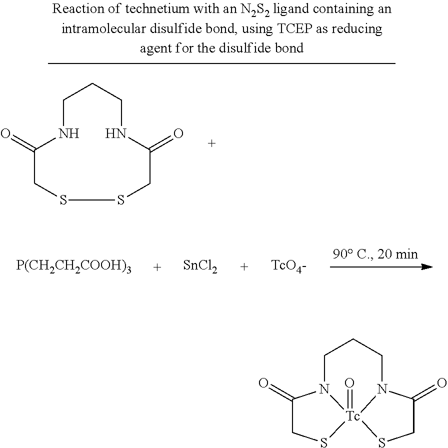

The diagram below illustrates the reaction of technetium with an N₂S₂ ligand containing an intermolecular disulfide bond using the phosphine compound TCEP as reducing agent for the disulfide bond.

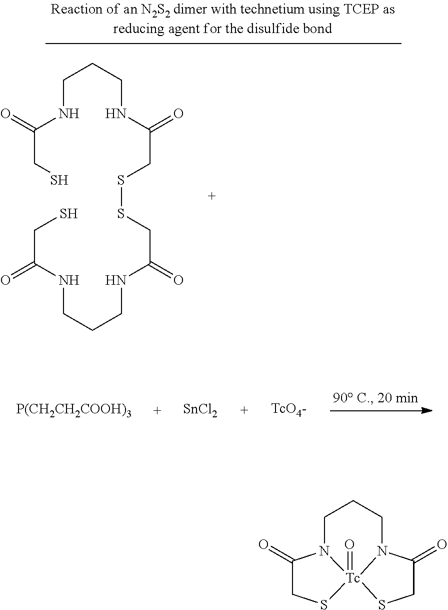

Formulations of the present invention prepared using phosphine compounds such as TCEP contain significantly less targeted chelating ligands than most prior art formulations. Indeed the formulations of the invention contain less than 10 μg of targeted chelating ligand per mL of diluent.

Most preferably, a radiopharmaceutical formulation prepared using a phosphine compound such as TCEP contains about 2 μg of targeted chelating ligand per ML of diluent. Formulations prepared using a phosphine compound such as TCEP may be prepared and administered to a subject without army purification.

The diluent used to prepare the radiopharmaceutical containing a phosphine compound such as TCEP may be any combination of water, normal saline and ethanol (EtOH), with the percentage of EtOH being preferably about 30%.

In a preferred embodiment of the present invention, a phosphine compound such as TCEP is added to a radiopharmaceutical formulation containing a compound of the following structure, together with sodium pertechnetate solution (^{99m}TcO₄⁻) and stannous gluconate and the mixture is heated at 90° C. for 20 minutes:

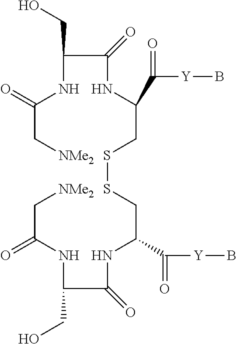

The resulting ^{99m}Tc labeled compound will exhibit an excellent radiochemical purity (RCP), indeed preferably the RCP is greater than 90% at six hours.

In a particularly preferred embodiment of the present invention, a phosphine compound such as TCEP is added to a radiopharmaceutical formulation containing compound 2, together with ^{99m}TcO₄⁻ and stannous gluconate and the mixture is heated at 90° C. for 20′.

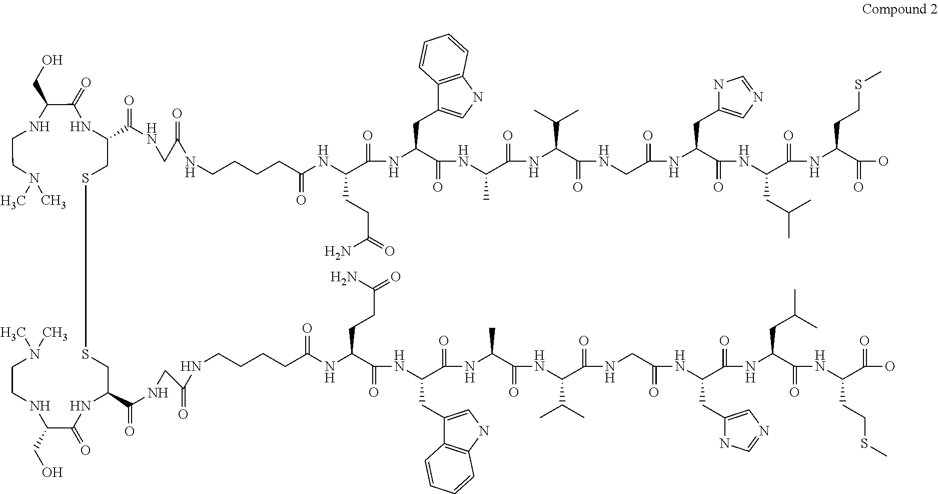

The results obtained, described in Example 6, demonstrate that the formulation containing TCEP that is used to prepare ^{99m}Tc Compound exhibits a radiochemical purity (RCP) greater than 90% at six hours. The ^{99m}Tc Compound 1 does not contain coordinated phosphine, as the retention time of the radiolabeled product was found to be identical to that obtained when prepared as described in WO 2003/092743, incorporated herein by reference in its entirety.

While the use of phosphines in reacting ligands with technetium is known, it has been typically observed that the phosphine will become coordinated to the Tc during the reaction. Phosphines are known to readily reduce technetium below Tc(V), to give oxidation states from Tc(I) to Tc(V), usually Tc(III). Thus, many phosphine containing complexes have been reported. Thus, it is unexpected that a phosphine compound could be used for the preparation of a radiopharmaceutical and not be incorporated into the resulting radiolabeled product.

The method of the present invention is unique in that the phosphine compound does not coordinate to the radiometal, when reacted with said radiometal in the presence of the novel precursor molecule or precursor compounds of the present invention.

### EXAMPLES

Unless otherwise noted, all materials were purchased from Aldrich and used without further purification. Fmoc-5-amino-valeric acid (5-ava) was purchased from Chem-Impex International. All Fmoc-amino acids were purchased from Novabiochem. Abbreviations used in the syntheses shown below are: TFA, trifluoroacetic acid; HOBt, 1-hydroxybenzotriazole; DIC, N,N′-diisopropylcarbodiimide; HATU, O-(7-azabenzotriazol-1-yl)-1,1,3,3-tetramethyl uronium hexafiuorophosphate; DIEA, diisopropylethylamine; DMF, dimethylformamide; DMSO, methyl sulfoxide. CH₂Cl₂, methylene chloride; EDC, N-(3-Dimethylaminopropyl)-N′-ethylcarbodiimide.

### Example 1

### Synthesis and Characterization of Compound 1 TFA Salt

Synthesis of compound 1 was carried out in dimethyl formamide (DMF) using HOBt/DIC activation on rink-amide Novagel resin. Fmoc deprotection was carried out with 20% piperidine DMF. The resin was swelled in DMF for 1 h before use. All couplings were of 2 hours duration except for the last N,N-dimethylglycine coupling (see below). The following scheme was used.

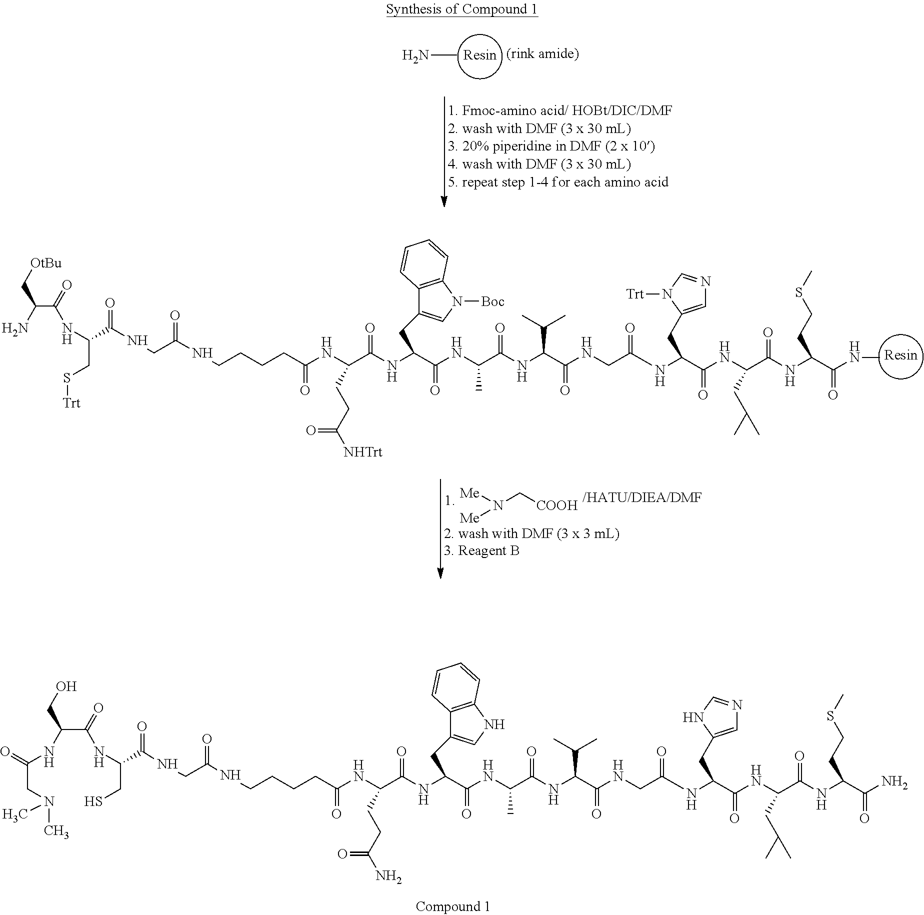

A typical coupling cycle is as follows: To a 50-mL SPPS reaction vessel containing 1.13 mmol of the swelled resin (0.6 mmol/g, Novabiochem) was added a solution of 4.52 mmol of an Fmoc-amino acid in DMF (EM Science), 4.52 mmol of HOBT (Novabiochem) in DMF, and 4.52 mmol of DIC. The total volume of DMF was 20 mL. The reaction mixture was shaken for 2 h. The resin was then filtered and washed with DMF (3×30 mL). A ninhydrin test was carried out to confirm the completion of the coupling. A solution of 20% piperidine in DMF (20 mL) was added to the resin and it was shaken for 10 min. The resin was filtered and this piperidine treatment was repeated. The resin was finally washed with DMF (3×30 mL) in preparation for the next coupling cycle.

At the last coupling cycle, N,N-d methyl glycine was coupled using HATU/DIEA activation. Thus, to a suspension of N,N-dimethyl glycine (4.52=top in DMF was added a solution of 4.52 mmol of HATU (Perseptive Biosystems) in DMF and 9.04 mmol of DIEA. The clear solution was added to the resin and shaken for 16 h. Following synthesis, the resin was washed with DMF (3×30 mL) and CH₂Cl₂ (3×30 mL). It was dried by blowing N₂ through the container for 15 min. Thirty mL of reagent B (prepared by mixing TFA [26 mL], phenol [1.5], /H₂O [1.5 mL] and triisopropylsilane [1.2 mL]) was added and it was shaken for 4 h. The resin was filtered and the filtrate was evaporated to a paste. The crude peptide was precipitated in diethyl ether and washed twice with ether. 1.2 g of the crude material was obtained after drying.

The thiol-containing peptide was purified using a Shimadzu HPLC system and a YMC C-18 preparative column. Crude material was dissolved in 15% CH₃CN/H₂O (0.1% TFA) and loaded on the column. The gradient consisted of an increase from 15% to 19% CH₃CN/H₂O (0.1% TFA) in 4 min., followed by 19% to 49% organic in 60 min. The fractions were combined and lyophilized. A total of 840 mg of the pure material was Obtained. The following analytical results were obtained.

Mass Spec: (M+H)⁺ at 1371.6; a doubly charged ion at 686.4.

HPLC: System 1: YMC C-18 (0.46×25 cm), UV at 220 nm, 15-55% CH₃CN/H₂O (0.1% TFA) in 20 min., retention time 13.90 min.

System 2: XTerra MS C-18 (0.46×50 mm), UV at 220, 10-40% CH₃CN/H₂O (0.1% TFA) in 8 min.; retention time 4.11 min.

Elemental Analysis: Found: C, 46.46; H, 5.81; N, 15.02, S, 4.31, H₂O 2.33; Calculated for C₆₀H₉₄N₁₈O₁₅S₂2TFA 2.4 H₂O: C, 46.79; H, 6.18; N, 15.35, S, 3.90, H₂O, 2.63.

Amino Add Analysis: Ser 0.2.9; Gly 1.38; Ala 0.70; Val 0.66; Met 0.58; Leu 0.72; H is 0.69.

### Example 2

### Preparation of Compound 2 from Compound 1

Compound 2, a disulfide dimer was prepared by aerial oxidation of Compound 1 following the procedure outlined below:

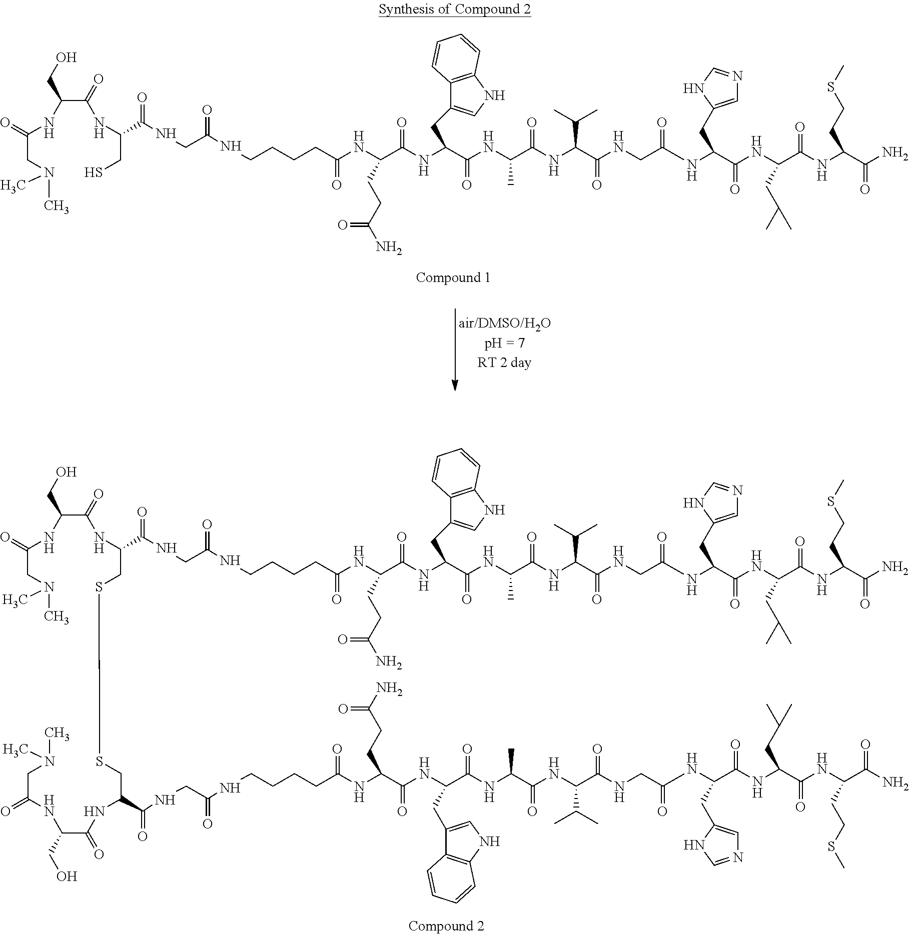

To a solution of 150 mg of Compound 1 in 2 mL of DMSO was added 40 mL, of H₂O (0.1% TFA). The pH of the clear solution was adjusted to 7 by adding saturated aqueous (NH₄)₂CO₃. It was stirred at RT in the open air for 2 days. The reaction was monitored by MS and HPLC to follow the progress of the oxidation. At completion, 30 mL of H₂O (0.1% TFA) was added to the cloudy mixture and it turned clear. It was loaded onto a YMC C-18 preparative HPLC column. The gradient was started at 5% CH₃CN/H₂O (0.1% TFA), increased to 14% organic in 9 min., then ramped from 14 to 34% organic in 80 min. The fractions containing desired product were lyophilized and a total of 93 mg of pure material was obtained. The analytical results for this compound are given below.

Mass Spec: a doubly charged ion at 1371.0; a triple charged ion at 914.7; a quadruple charged ion at 686.3.

### HPLC

System 1: YMC C-18 (0.46×25), UV at 220 nm, 15-55% CH₃CN/H₂O (0.1% TEA) in 20 min., retention time 15.31 min.

### Example 3

### Synthesis of Compound 9

The following scheme was used to prepare Compound 9.

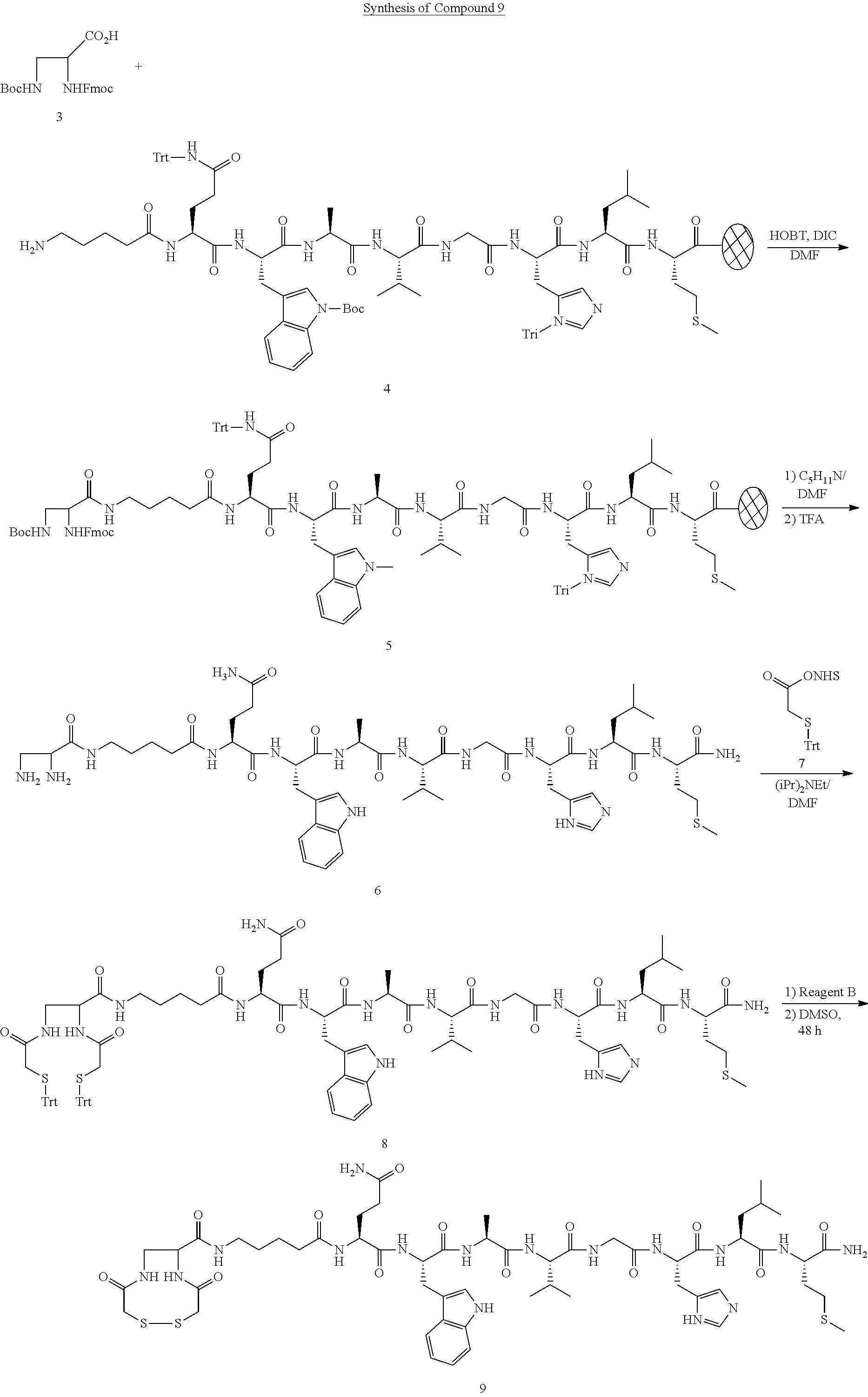

Peptide (4): Compound 3 was obtained from Bachem. Synthesis of peptide 4 was carried out on a 0.25 mmol scale using an ABI 433 A synthesizer with the FastMoc protocol (Applied Biosystems Inc.). The peptide was made using 0.4 g of Rink amide Nova Gel HL resin, (resin substitution 0.6 mmol/g).

In each cycle of this protocol, 1.0 mmol of a dry protected amino acid in a cartridge was dissolved in a solution of 0.9 mmol of HBTU, 2 mmol of DIEA, and 0.9 mmol of HOBt in DMF with additional NMP added. The coupling time in this protocol was 21 min. Peptide loaded resin 4 (0.7 g) was obtained from ABI synthesizer. Fmoc deprotection was carried out with 20% piperidine in DMF (2×10 mL) for 10 min. The peptide bound resin 4 was washed with DMF (3×10 mL) and CH₂Cl₂ (3×10 mL) and dried.

Compound (7) (Luyt et al. Bioconjugate Chem., 1999, Vol 10, 470-479): Concentrated sulfuric acid (5 g) was added to a solution of mercaptoacetic acid (4.6 g, 0.05 mmol) and trityl alcohol (9.13 g, 0.05 mol) and the mixture was stirred at 70° C. for 12 h. Acetic acid was removed under vacuum and the residue was poured into ice. The solid formed was filtered and dried under vacuum. 2,4,6-collidine (1.2 g, 0.01 mol) was added to a mixture of S-tritylmercaptoacetic acid (3.34 g, 0.01 mol), N-hydroxysticcinimide (1.15 g, 0.01 mol) and EDC (1.91 g, 0.01 mol) in acetonitrile (50 mL) and the mixture was stirred at RT for 6 h. Acetonitrile was removed and the residue was poured into water, extracted with ethyl acetate and dried (Na₂SO₄). Concentration of the ethyl acetate solution gave a solid, which was purified by silica gel column chromatography using hexane/ethyl acetate (7/3). Fractions containing the product were collected and concentrated to give a white solid. Yield 3.82 g (88%). MS: 431.0 (M+H)

Compound (6). Peptide loaded resin 4 (0.4 g, 0.24 mmol) was reacted with N-α-Fmoc-N-β-t-Boc-L-diaminopropionic acid (0.43 g, 1 mmol), HOBT (0.153 g, 1 mmol) and DIC (0.13 g, 1 mmol) in 10 mL of DMF for 6 h. The resin was then washed with DMF (2×10 mL). The Fmoc group was removed using 20% piperidine in DMF. The peptide bound resin was washed with DMF (3×10 mL) and CH₂Cl₂ (3×10 mL), dried and cleaved from the resin (using reagent B: TFA/triisopropylsilane/phenol/H₂O 8.6 mL, 0.4 mL, 0.5 g, 0.5 mL), TFA was removed and the residue was triturated with ether. The precipitated peptide 6 was filtered and dried. Yield 220 mg (80%). MS: 1125.6 (M+H).

Compound (9). Diisopropylethylamine (100 μL) was added to a mixture of the peptide 6 (0.112 g, 0.09 mmol) and S-tritylmercaptoacetic acid N-hydroxy-succinimide ester 7 (100 mg, 0.23 mmol) in DMF (0.5 mL) and stirred for 6 h. The progress of the reaction was monitored by HPLC. After the reaction was complete, DMF was removed under vacuum and the residue was treated with reagent B (0.5 mL) and stirred for 3 h. TFA was removed and the residue was triturated with dry ether (5.0 mL). The precipitated white solid was filtered and, dried under vacuum. Yield 80 mg (87%). The deprotected peptide (80 mg, 0.06 mmol) was dissolved, in DMSO (1.0 mL) and the mixture was stirred for 48 h. The cyclization was followed by HPLC. After complete cyclization the DMSO solution was directly purified by preparative HPLC. Fractions containing the pure peptide were pooled and freeze dried to yield the cyclic peptide 9. Yield 22 mg (25%). MS: 1293.4 (M+Na), 1271 (M+H), 674.2 (M+Na+H)/2, 658.5, (M+2Na)/2.

Retention Time: 5.23 min; Analytical purity. 96.0%; Column: Waters XTerra MSC18, 4.6×50 mm; Particle size: 5 microns; Eluents: A: Water (0.1% TEA), B: Acetonitrile (0.1% TEA); Elution: Initial Conditions: 10% B, linear gradient 1-100% B in 10 min; Flow rate: 3 mL/min; Detection: UV @ 220 nm.

### Example 4

### Syntheses of Compound 14 and Compound 15

The following scheme was used to prepare Compounds 14 and 15.

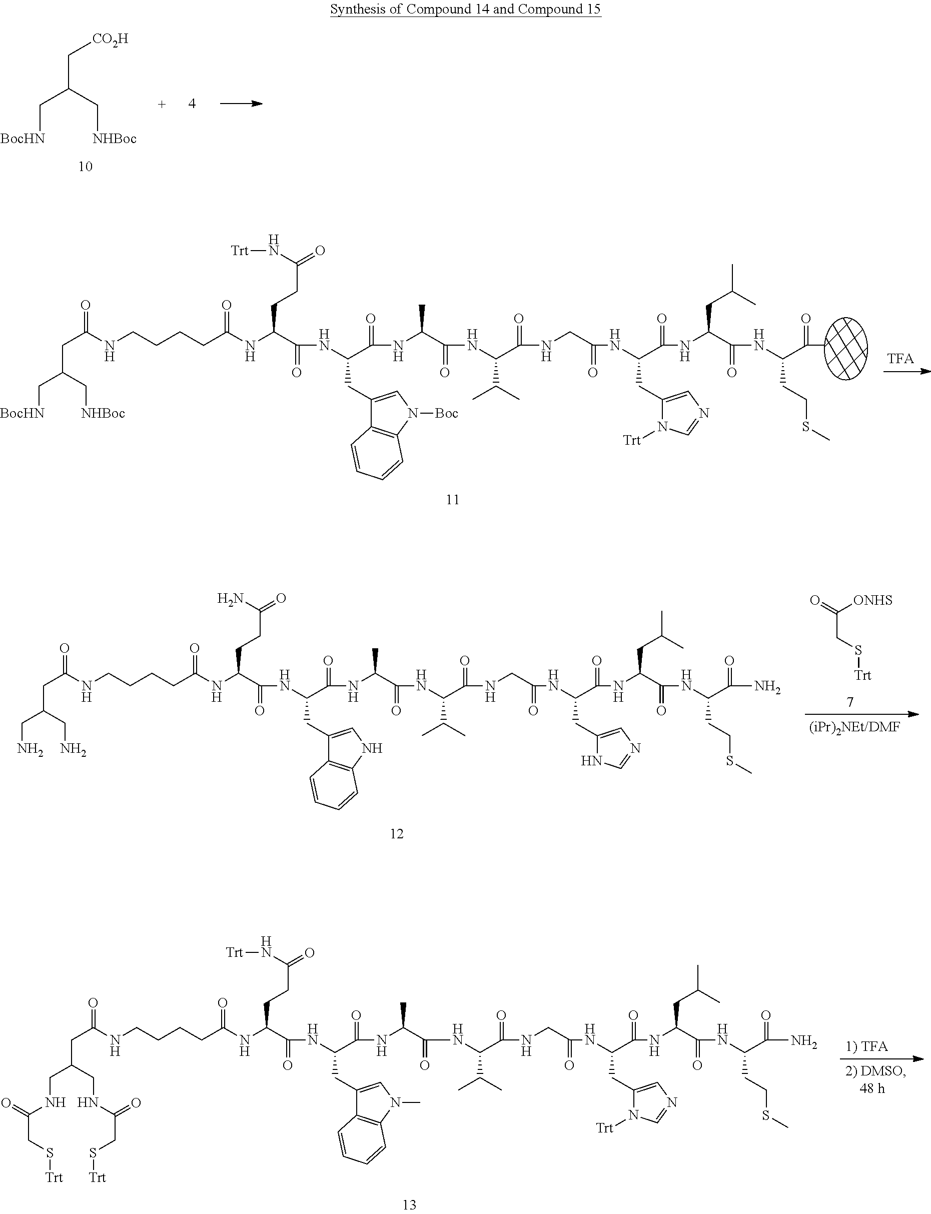

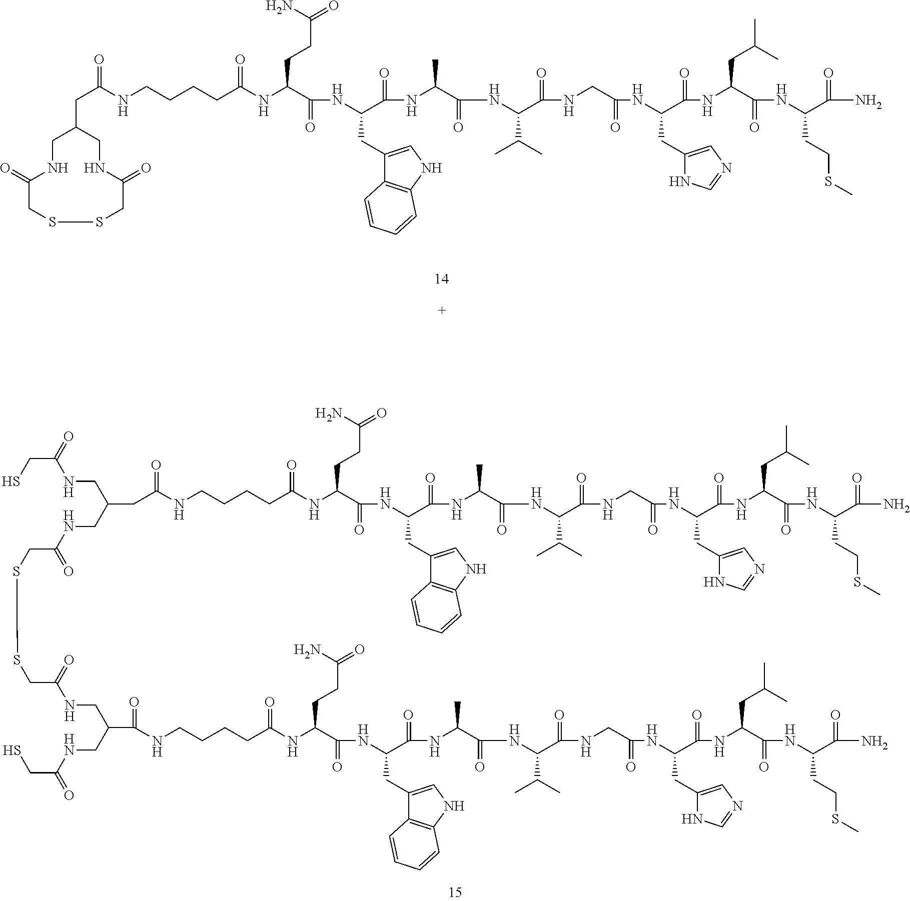

Compound (12). Peptide bound resin 4 (0.3 g, 0.18 mmol) was reacted with di-t-boc acid 10 (Albrecht et al. Pept.: Chem., Struct. Biol., Proc. Am. Pept. Symp., 11th (1990), Meeting Date 1989, 718-20)(100 mg, 0.3 mmol) in DMF (2.0 mL) in the presence of HOBT (45 mg, 0.3 mmol) and DIC (39 mg, 50 μL, 0.3 mmol) in DMF (10 mL). The reaction was carried out for 6 h. After the reaction the resin was washed with DMF (3×10 mL) and CH₂Cl₂ (3×10 mL) and dried under vacuum. The peptide was cleaved from the resin using reagent B (10.0 mL) for 4 h. TFA solution was filtered and concentrated to give pasty solid. The solid was triturated with ether and the resulting peptide 12 was collected by filtration and dried under vacuum. Yield 150 mg (72%). MS: 1153.4 (M+H).

Compounds (14) and (15). Diisopropylethylamine (100 μL) was added to a mixture of the peptide 12 (0.115 g, 0.1 mmol) and S-tritylmercaptoacetic acid N-hydroxysuccinimide ester 10 (100 mg, 0.23 mmol) in DMF (2.0 mL) and stirred for 6 h. DMF was removed under vacuum and Reagent 13 (3.0 mL) was added to the residue and the mixture was stirred for 4 h. TFA was removed and the resulting pasty solid was triturated with ether (10.0 mL). The precipitated solid was filtered and dried under vacuum. Yield 95 mg (77%). The deprotected peptide (95 mg, 0.08 mmol) was dissolved in DMSO and stirred for 8 h. The progress of the reaction was followed by HPLC. Two products were observed. One is due to the cyclic compound and the other due to the linear dimer. The DMSO solution was then purified by preparative HPLC and the two products were collected. Fractions were then pooled and freeze dried to give compound 14 (15 mg, 14%) and compound 15 (22 mg, 21%).

Compound 14, MS: 1321.4 (M+Na), 1299.4 (M+H), 672.8 (M+2Na)/2, 661 (M+H+Na)/2, 650.6 (M+2E1)/2. Retention time: 4.08 min; Analytical purity: 98.0%; Column: Waters XTerra MSC18 4.6×50 mm; Particle size: 5 microns; Eluents: A Water (0.1% TFA), B′. Acetonitrile (0.1% TFA); Elution: initial Condition: 10% B, linear gradient 1-100% B in 10 min; How rate: 3 mL mm, Detection: UV 220 nm.

Compound 15. MS: 1321.9 (M+2Na)/2, 1311.5 (M+Na)/2, 1300.1 (M+H)/2, 867 (M+3H)/3, 874.8 (M+Na)/3, Retention time: 4.57 min; Analytical purity 98.0° X); Column: Waters XTerra MSC18 4.6×50 nm; Particle size: 5 microns; Eluents: A: Water (0.1% TFA), B: Acetonitrile (0.1% TFA); Elution: Initial Condition: 10% B, linear gradient 10-100% B in 10 min; Flow rate: 3 mL/min; Detection: UV @220 nm.

### Example 5

### Syntheses of ^{99m}Tc Compound 1

Stannous gluconate solutions were prepared by dissolving SnCl₂.2H₂O (2.1-4.9 mg) in N₂-purged 0.05 N HCl (250-500 pt). The volume was adjusted with N₂-purged H₂O (2.62-6.12 mL) and sodium gluconate (139-323 mg) was added. Compound 2 (149-311 μg) was dissolved in H₂O (1.8-4.47 mL). TCEP (2.1-3.9 mg) was dissolved in 10 mL of H₂O; 1 mL of this solution was further diluted with H₂O (2.42-5.25 mL). Ascorbic acid (85.6-102.7 mg) was dissolved in H₂O (2.14-7.77 mL), maltose (169.7-184.3 mg) was dissolved in H₂O (1.84-2.8 mL) and hydroxypropyl-γ-cyclodextrin (Hp-γ-CD) (130.3 mg) was dissolved in 1.30 mL of H₂O.

To 50 μL (2 μg) of a solution of Compound 2 was added 200 μL of ^{99m}TcO₄⁻ (34.6 to 43 mCi), H₂O (100-200 μL), 50 μL of the stannous gluconate solution (40 μg of SnCl₂), 300 μL of ethanol, 50 μL (6 μg) of the TCEP solution and 100 μL of 0.1M NH₄Ac pH 5 buffer. The reaction was heated at 90° C. for 20 min. and then 50 μL (2 to 3 mg) of the ascorbic acid solution, 0 to 50 μL (0 to 5 mg) of the maltose solution and 0 to 50 μL (0 to 5 mg) of the Hp-γ-CD solution were added.

HPLC analysis was performed at 0 and 6 h using the following system: Vydac C-18 Protein and Peptide column (4.6×250 mm) eluted with 77% H₂O (0.1% TFA)/23% CH₃CN (0.1% TFA) for 30 mM. Flow rate: 1 mL/min. The resulting RCP ranged from 90.9 to 96.2% initially and from 90.5 to 93.4% at 6 hours (n=5).

### Example 6

### Syntheses of ^{99m}Tc Compound 15

A stannous gluconate solution was prepared by dissolving 4.745 mg (0.021 mmol) of SnCl₂.2H₂O in 500 μL of N₂-purged 0.01 N HCl. The volume was adjusted to 1.76 mL with N₂-purged H₂O and 156 mg (0.714 mmol) of sodium gluconate was added. Compound 15 (217 μg) was dissolved in 1.35 mL of 15% DMF/85% H₂O. TCEP (1.572 mg) was dissolved in 4.37 mL of H₂O.

To 50 μL (4 μg) of Compound 15 solution was added 25 μL of ^{99m}TcO₄⁻ (2 mCi), 390 μL of H₂O, 20 μL of the stannous gluconate solution (40 μg of SnCl₂), 385 μL of EtOH, 50 μL (18 μg) of the TCEP solution and 200 μL of 0.1M sodium carbonate buffer, 9. The reaction was heated at 100° C. for 15 minutes prior to HPLC analysis that was performed at 0 and 6 h. using the following system: Vydac C-18 Protein and Peptide column (4.6×250 mm) eluted as follows: from 78% H₂O (0.1% TFA)/22% CH₃CN (0.1% TFA) to 70%/30% in 20 min; from 70%/30% to 60%/40% in 5 min, hold for 10 Min, from 60/40 to 78/22 in 5 min, Flow rate 1 mL/min. The resulting RCP was 41.5% initially and 42.2% at 6 hours.

### Example 7

### Synthesis of ^{99m}Tc Compound 14

To 50 μL (4 μg) of Compound 14 solution was added 22 μL of ^{99m}TcO₄⁻ (1.9 mCi), 388 μL of H₂O, 20 μL of the stannous gluconate solution (40 μg of SnCl₂), 300 μL of EtOH, 50 μL (18 μg) of the TCEP solution and 200 μL of 0.1M sodium carbonate buffer, pH 9. The reaction was heated at 100° C. for 15 minutes prior to HPLC analysis that was performed at 0 and 6 h using the system described in Example 6. The resulting RCP was 44.9% initially and 46.5% at 6 hours.

### Example 8

### Synthesis of ^{99m} Tc Compound 9

To 50 μL (4 μg) of Compound 9 solution was added 20 μL of ^{99m}TcO₄⁻ (2 mCi), 390 μL of H₂O, 20 μL of the stannous gluconate solution (40 μg of SnCl₂), 300 μL of EtOH, 50 μL (18 μg) of the TCEP solution and 200 μL of 0.1M sodium carbonate buffer, pH 9. The reaction was heated at 100° C. for 15 minutes prior to HPLC analysis that was performed at 0 and 6 h using the HPLC system described in Example 6. The resulting RCP was 61.5% initially and 60.5% at 6 hours.

1-38. (canceled)

39. A method of complexing a metal to a chelating group comprising at least one thiol, said method comprising the following steps:
(i) providing a disulfide-containing precursor molecule in accordance with claim 1, wherein said thiol is bound to a second thiol forming an intermolecular disulfide bond in the precursor molecule; and
(ii) reducing said disulfide bond by treating said precursor molecule with a phosphine compound in the presence of said metal, thereby forming said complex.

40. (canceled)

41. (canceled)

42. The method of claim 39, wherein said second thiol group is present on another molecule of the same compound.

43. The method of claim 39, wherein said second thiol group is present on a molecule of a different compound.

44. The method of claim 39, wherein said metal is selected from the group consisting of transition metals, lanthanides, auger-electron emitting isotopes, and α-, β- or γ-emitting isotopes.

45. The method of claim 44, wherein the metal is selected from the group consisting of: ⁶⁴Cu, ⁶⁷Cu, ⁶⁷Ga, ¹⁰⁵Rh, ^{99m}Tc, ^{186/188}Re, ¹⁵³Sm, ¹⁶⁶Ho, ¹¹¹In, ⁹⁰Y, ¹⁷⁷Lu, ¹⁰⁹Pd, ¹⁴⁹Pm, ¹⁶⁶Dy, ¹⁷⁵Yb, ¹⁹⁹Au and ^{117m}Sn.

46. The method of claim 44, wherein said metal is an isotope of Tc.

47. The method of claim 39, wherein said phosphine compound is selected from the group consisting of m,m,m-trisulfonatetriphenylphosphine (TPPTS), m,m,disulfonatetriphenylphosphine (TPPDS), Tris(dimethylamino)phosphine, Tris(hydroxymethyl)phosphine and Tris[2-carboxyethyl]phosphine (TCEP).

48. The method of claim 47, wherein said phosphine compound is TCEP.

49. The method of claim 39 resulting in at least 90% yield of said complex.

50. The method of claim 39, wherein step (ii) is carried out in the presence of a stannous compound.

51. The method of claim 50, wherein step (ii) is carried out in the presence of ethanol and sodium bicarbonate buffer having a pH of about 9.

52. The method of claim 39, wherein step (ii) is carried out at a temperature from about 70° C. to about 100° C.

53. The method of claim 52, wherein step (ii) is carried out at a temperature of about 100° C.

54. The method of claim 39, wherein said precursor compound or each of the compounds linked by disulfide bonds in said precursor molecule has a structure of the formula X-Y-B, wherein:
(a) X is the metal chelating group containing said thiol group;
(b) Y is a spacer group or covalent bond; and
(c) B is a targeting group.

55. The method of claim 54, wherein X is selected from the group consisting of BAT, DADS, MAG3, CODADS, N₃S, N₂S₂, NS₃ and derivatives thereof.

56. The method of claim 54, wherein X in the compounds linked by disulfide bonds in said precursor molecule is BAT or a derivative thereof.

57. The method of claim 54, wherein X in the compounds linked by disulfide bonds in said precursor molecule is N₃S or a derivative thereof.

58. The method of claim 54, wherein X in the compounds linked by disulfide bonds in said precursor molecule is a monoamine bis amide monothiol (N₃S).

59. The method of claim 54, wherein X in the compounds linked by disulfide bonds in said precursor molecule is N,N-dimethylGlycine-Ser-Cys (N₃S).

60. The method of claim 54, wherein X in the compounds linked by disulfide bonds in said precursor molecule is N,N-dimethylGlycine-Thr-Cys (N₃S).

61. The method of claim 54, wherein X in said precursor compound is N₂S₂ or a derivative thereof.

62. The method of claim 54, wherein said targeting group is a peptide.

63. The method of claim 54, wherein said targeting group is a gastrin releasing peptide (GRP) receptor agonist.

64. The method of claim 63, wherein said targeting group is selected from the group consisting of BBN(7-14) and BBN(8-14).

65. The method of claim 54, wherein Y is selected from the group consisting of at least one amino acid residue, a hydrocarbon chain and a combination thereof.

66. The method of claim 65, wherein Y is selected from the group consisting of glycine, β-alanine, gamma-aminobutanoic acid, 5-aminovaleric acid (5-Ava), 6-aminohexanoic acid, 7-aminoheptanoic acid, 8-aminooctanoic acid (8-Aoc), 9-aminononanoic acid, 10-aminodecanoic acid and 11-aminoundecanoic acid (11-Aun).

67. The method of claim 65, wherein Y is Gly-Ser-Gly.

68. The method of claim 54, wherein said metal chelating group binds a metal selected from the group consisting of transition metals, lanthanides, auger-electron emitting isotopes, and α-, β- or γ-emitting isotopes.

69. The method of claim 68, wherein the metal is selected from the group consisting of: ⁶⁴Cu, ⁶⁷Cu, ⁶⁷Ga, ⁶⁸Ga, ¹⁰⁵Rh, ^{94m}Tc, ^{99m}Tc, ^{186/188}Re, ¹⁵³Sm, ¹⁶⁶Ho, ¹¹¹In, ⁹⁰Y, ¹⁷⁷Lu, ¹⁰⁹Pd, ¹⁴⁹Pm, ¹⁶⁶Dy, ¹⁷⁵Yb, ¹⁹⁹Au and ^{117m}Sn.

70. The method of claim 68, wherein the metal is an isotope of Tc.

71. A method of complexing a metal to a thiol group, said method comprising the following steps:
providing a disulfide-containing precursor compound, wherein said thiol is bound to a second thiol forming an intermolecular disulfide bond; and
(ii) reducing said disulfide bond by treating said precursor compound with a phosphine compound in the presence of said metal, thereby forming said complex.

72-77. (canceled)
